(19) United States
(12) Patent Application Publication
(10) Pub. No.: US 2019/0119645 A1
(43) Pub. Date: Apr. 25, 2019

(54) GENERATION OF ADULT-LIKE HEART MUSCLE FROM HUMAN PLURIPOTENT STEM CELLS

(71) Applicant: The Johns Hopkins University, Baitimore, MD (US)

(72) Inventors: Chulan Kwon, Ellicott CIty, MD (US); Gunsik Cho, Balitmore, MD (US)

(21) Appl. No.: 15/771,925

(22) PCT Filed: Oct. 27, 2016
(86) PCT No.: PCT/US2016/059073

§ 371 Date: Jan. 3, 2019

Related U.S. Application Data
(60) Provisional application No. 62/246,727, filed on Oct. 27, 2015.

(51) Int. Cl. C12N 5/077; C12N 5/0793; G01N 33/50; A61K 35/34; A61K 35/30

(52) U.S. Cl. CPC C12N 5/0657; C12N 5/0619; C12N 2506/45; A61K 35/34; A61K 35/30; G01N 33/5008

(57) ABSTRACT

The present invention relates to compositions and methods for generating adult cells from pluripotent stem cells. The invention also relates to methods of treating disease using adult cells derived from pluripotent stem cells. The resulting adult cells are used for drug testing, drug screening, and regeneration therapy to treat heart diseases and neurodegenerative diseases.

### RELATED APPLICATIONS

This application claims the benefit of priority under 35 U.S.C. § 119(e) to U.S. Provisional Application No. 62/246,727, filed Oct. 27, 2015, which is incorporated herein by reference in its entirety.

### STATEMENT REGARDING FEDERALLY SPONSORED RESEARCH OR DEVELOPMENT

This invention was made with government support under RO1HL111198 awarded by the National Institutes of Health (NIH), under HL119012 awarded by the National Health Service (NHLBI), and HL107153 awarded by the National Health Service (NHLBI). The government has certain rights in the invention.

### BACKGROUND OF THE INVENTION

Pluripotent stem cells (PSCs) are capable of becoming all types of patients' body cells and thus, provide unprecedented opportunities for disease modeling and personalized medicine. With the capability, cardiac regenerative medicine focuses on utilizing PSCs for modeling heart disease and cell-based interventions of cardiac repair. However, prior to the invention described herein, PSC-derived cardiomyocytes exhibited fetal-like characteristics, and the immaturity remained a key impediment for their therapeutic applications for late-onset diseases. As such, there was a pressing need to develop methods to generate adult cardiomyocytes from human PSCs, which allow modeling, drug testing, and treating adult heart diseases with patients' own PSCs.

### SUMMARY OF THE INVENTION

The present invention is based, at least in part, upon the identification of methods to generate adult cells from pluripotent stem cells (PSC) in a relatively short period of time, e.g., in about 4-6 weeks. Specifically, the PSCs described herein are incubated with differentiated tissue (e.g., a postnatal cellular environment) to generate adult cells. In some embodiments, adult cells are generated by incubation of PSCs in organs from other mammalian species. For example, human adult cardiomyocytes are produced by incubating human PSC-induced cardiomyocytes or cardiac progenitor cells (CPCs) in postnatal rodent hearts. Furthermore, as described herein, adult cells produced by the methods provided herein are used to treat diseases or conditions, such as those that manifest later in life. In some embodiments, adult cells generated by the methods described herein are used to model and treat diseases or conditions in a subject such as cardiac-related disorders (e.g., cardiomyopathy), neurodegenerative disorders (e.g., Parkinson's disease and Alzheimer's disease), muscle-related disorders (e.g., Muscular Dystrophy), liver disorders (e.g., fatty liver disease or hepatitis), lung disorders (e.g., asthma), and pancreas disorders (e.g., pancreatitis). The compositions described herein are also utilized in drug screening methods for treatments of different diseases or disorders. The subject is preferably a mammal in need of such treatment, e.g., a subject that has been diagnosed with a cardiac-related or neurodegenerative or muscle-related disease or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Provided herein are methods of producing an adult cell from a pluripotent stem cell (PSC) comprising: obtaining/providing a PSC; inducing a PSC to become an immature PSC-derived cell, contacting a PSC-derived cell, e.g., generated in vitro, with a postnatal cellular environment (e.g., a postnatal heart) for a period of time, maturing the PSC-derived cells into an adult cell, thereby producing an adult cell from a pluripotent stem cell.

This system is a diagnostic tool to predict pathogenesis of late-onset disease (e.g., cardiomyopathy, Parkinson's disease, and Alzheimer's disease) in a patient-specific manner. Late-onset/adult diseases manifest in mature cell types. To model this, patient-derived human iPSCs are differentiated into progenitor cells of desired tissues/organs in vitro and matured in the tissues/organs of animals from early postnatal stages to become adult human cells using the methods described herein. The resulting adult cells recapitulate disease phenotype and can be analyzed in vivo and in vitro. In addition, by monitoring and tracing the maturation of these cells, this method allows for identifying earliest disease onset with bioinformatics such as ribonucleic acid sequencing (RNA-seq) analysis. For example, methods of diagnosing/predicting disease pathogenesis are carried out by detecting a biomarker of disease progression in an adult cell produced by the methods described herein. For example, RNA-seq is utilized to detect the biomarker.

Moreover, the adult cells produced by the methods described herein can be directly used to mimic a clinical trial with potential drugs for personalized/precision medicine. A number of drugs which are effective in animals fail in human clinical trials (>90%). Prior to the invention described herein, human iPSC-derived cells remained fetal-like in vitro, thereby rendering them useless in drug testing of adult onset diseases. The methods described herein allow for the generation of human adult cells/tissues in animals. Accordingly, the resulting animals with humanized cells/tissues/organs are used to mimic human clinical trials or to test drug efficacy for personalized medicine. For example, methods of determining efficacy of a candidate compound for treatment of a disease are carried out by administering a candidate compound to adult cells produced by the methods described herein and determining whether the disease is inhibited by the candidate compound, thereby determining efficacy of the candidate compound for treatment of the disease. In some cases, the adult cells are present in a mammal selected from a group consisting of rodents, rats, mice, rabbits, goats, non-human primates, humans, dogs, bears, cats, lions, tigers, elephants, llamas, donkeys, mules, bovines, ovines, pigs, and horses.

Optionally, the adult cell is isolated for further use such as cell-based regenerative therapy. Method of administering regenerative therapy to a subject are carried out by administering to a subject in need thereof an adult cell produced by the methods described herein.

In some cases, the PSC are induced to become an immature PSC-derived cell (e.g., an immature cardiomyocyte) in an in vitro setting (e.g., a dish) prior to contact with the postnatal cellular environment.

In some cases, the postnatal cellular environment comprises a cell selected from the group consisting of a cardiomyocyte, a cardiac conduction cell, a hepatocyte, a neuron, a leukocyte, an astrocyte, a brain cell, a photoreceptor cell, a retinal cell, a lung cell, a kidney cell, a pancreatic cell, a lymphocyte, a T cell, a B cell, a chondrocyte, an osteoblast, a skeletal muscle cell, a spleen cell, a stomach cell, an intestinal cell, a bladder cell, and skin cell.

In some cases, the period of time for contacting a PSC-derived cell with the postnatal cellular environment is from about 1 hour to about 6 months, e.g., about 1 day to about 4 months, e.g., about 1 week to about 8 weeks, e.g., 1 week, 2 weeks, 3 weeks, 4 weeks, 5 weeks, 6 weeks, 7 weeks, or 8 weeks. Preferably, the period of time is about 4 to 6 weeks.

In some cases, the postnatal cellular environment comprises a cell from a postnatal heart. Preferably, the postnatal heart is less than a 4 week old postnatal heart, e.g., less than 3 week old, less than 2 week old, or less than one week old postnatal heart. Preferably, the postnatal heart is less than 2 weeks old.

In some cases, the PSC-derived cell is contacted with the 2 week postnatal heart via intraventricular delivery of the cell into the heart. For example, 1×10² to 1×10⁹ PSC-derived cells are injected into the postnatal heart, e.g., 1×10³, 1×10⁴, 1×10⁵, 1×10⁶, 1×10⁷, or 1×10⁸ PSC-derived cells are injected into the postnatal heart. In one aspect, about 2×10⁵ PSC-derived cells are injected into the postnatal heart. Exemplary numbers of PSC-derived cells include 200,000 PSC-derived cells per injection with 2,000 adult cells obtained (i.e., 1% overall engraftment). Optionally, multiple injections can be performed if more adult cells are needed. While conventional injection methods are used, other methods can be used to improve engraftment. In some cases, the PSC is a human PSC. In some cases, the adult cell is a cardiomyocyte, i.e., the PSC-derived immature cardiomyocyte matures into a cardiomyocyte in the postnatal heart.

In some cases, the PSC-derived cell is contacted with a postnatal cellular environment from a mammal. For example, the mammal is a rodent, a rat, a mouse, a rabbit, a goat, a non-human primate, a human, a dog, a bear, a cat, a lion, a tiger, an elephant, a llama, a donkey, a mule, a bovine, an ovine, a pig, or a horse.

Also provided herein are methods of treating a disease or condition comprising administering to a subject suffering from or at risk of suffering from the disease or condition an adult cell produced by contacting a PSC-derived cell with the postnatal cellular environment for a period of time and maturing the PSC-derived cells into an adult cell. In some cases, the adult cells are generated by incubating PSC-derived cells with differentiated tissue (e.g., the postnatal cellular environment) of the same cell type as the diseased tissue for a suitable period of time until the PSC-derived cells have matured into adult cells.

In some cases, the disease is selected from a group consisting of a cardiac disorder, an immune disorder, a cancer, a gastro-intestinal disorder, a neurological disorder, a neurodegenerative disorder, a skeletal disorder, and a pulmonary disorder. In some cases, the disease is a cardiac disorder. Exemplary cardiac disorders include cardiovascular disease, cardiomyopathy, atherosclerosis, myocardial infarction, stroke, endocarditis, rheumatic heart disease, hypertensive heart disease, and angina. Preferably, the cardiac disorder is cardiomyopathy.

In some cases, the disease is a neurodegenerative disorder. Exemplary neurodegenerative disorders include Alzheimer's disease, Parkinson's disease, amyotrophic lateral sclerosis (ALS), Huntington's disease, spinocerebellar ataxia type 1 (SCA1), and prion-disorder. Preferably, the neurodegenerative disorder is Parkinson's disease or Alzheimer's disease.

Also provided herein are methods of treating a cardiac-related disease or condition comprising administering to a subject suffering from or at risk of suffering from a cardiac-related disease or condition an adult cell produced by contacting a PSC-derived cell with the postnatal cellular environment for a period of time and maturing the PSCs into an adult cell.

In some cases, the postnatal cellular environment is one or more postnatal heart cells from a mammal, e.g., an “early” postnatal heart cell. For example, the postnatal heart cell is less than a 4 week old postnatal heart cell, e.g., less than 3 week old, less than 2 week old, or less than one week old postnatal heart cell. In some cases, the adult cells are cardiomyocytes. In one aspect, the cardiac-related disease is cardiomyopathy.

Also provided are methods of treating a neurodegenerative disease or condition comprising administering to a subject suffering from or at risk of suffering from a neurodegenerative disease or condition an adult cell produced by contacting a PSC with a differentiated cell for a period of time and maturing the PSC into an adult cell.

In one aspect, the postnatal cellular environment is one or more postnatal neurons from a mammal, e.g., “early” postnatal neurons. For example, the postnatal neuron cell is less than a 4 week old postnatal neuron cell, e.g., less than 3 week old, less than 2 week old, or less than one week old postnatal neuron cell. In some cases, the adult cells are neurons. Exemplary neurodegenerative diseases include Parkinson's disease and Alzheimer's disease.

Also provided are compositions comprising an adult cell produced by contacting a PSC with the postnatal cellular environment for a period of time; maturing the PSCs into an adult cell, thereby producing an adult cell from a pluripotent stem cell. In some cases, PSC-derived cells are labeled with markers selected for identification. Suitable markers include fluorescent markers, e.g., fluorescent proteins, and bacterial proteins. In other cases, cells are genetically labeled. In one aspect, labeled cells are identified and /or isolated after maturation. Alternatively, PSC-derived cells are distinguished by species-specific antibodies.

In some cases, during the treatment methods described herein, the adult cell is administered to a subject in need thereof via oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, intraventricular injection, injection into the brain or spinal cord, or subcutaneous administration.

Finally, the methods described herein are useful in predicting/diagnosing future disease. Since many animals have shorter life spans compared to humans, (e.g., 1-2 months to adulthood in rodents), and because the engrafted cells are matured and aged along with the animals' own cells, the methods described herein allow for acceleration of potential disease in cells, tissues, and organs. In this manner, the methods described herein allow practitioners to “see the future” of cells, tissues, and organs, thereby affording the opportunity for early treatment options. Accordingly, methods of diagnosing/predicting disease are carried out by detecting a biomarker of disease in an adult cell produced by the methods described herein. For example, RNA-seq is utilized to detect the biomarker.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “binding to” a molecule is meant having a physicochemical affinity for that molecule.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence or amount of the analyte to be detected.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., prostate cancer, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

By “modulate” is meant alter (increase or decrease). Such alterations are detected by standard art known methods such as those described herein.

The term, “normal amount” refers to a normal amount of a complex in an individual known not to be diagnosed with a disease or disorder. The amount of the molecule can be measured in a test sample and compared to the “normal control level,” utilizing techniques such as reference limits, discrimination limits, or risk defining thresholds to define cutoff points and abnormal values (e.g., for prostate cancer). The “normal control level” means the level of one or more proteins (or nucleic acids) or combined protein indices (or combined nucleic acid indices) typically found in a subject known not to be suffering from prostate cancer. Such normal control levels and cutoff points may vary based on whether a molecule is used alone or in a formula combining other proteins into an index. Alternatively, the normal control level can be a database of protein patterns from previously tested subjects who did not convert to a disease or disorder over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

A “purified” or “biologically pure” nucleic acid or protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “pluripotency” or “pluripotent stem cells” is meant stem cells with the potential to differentiate into any of the three germ layers: endoderm (interior stomach lining, gastrointestinal tract, the lungs), mesoderm (muscle, bone, blood, urogenital), or ectoderm (epidermal tissues and nervous system). However, cell pluripotency is a continuum, ranging from the completely pluripotent cell that can form every cell of the embryo proper, e.g., embyronic stem cells, to the incompletely or partially pluripotent cell that can form cells of all three germ layers, but that may not exhibit all the characteristics of completely pluripotent cells.

By “stem cells” is meant undifferentiated biological cells that can differentiate into specialized cells and can divide (e.g., through mitosis) to produce more stem cells. They are found in multicellular organisms. In mammals, there are two broad types of stem cells: embryonic stem cells, which are isolated from the inner cell mass of blastocysts, and adult stem cells, which are found in various tissues.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

By “reference” is meant a standard or control condition.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

As used herein, “obtaining” as in “obtaining an agent” includes synthesizing, purchasing, or otherwise acquiring the agent.

The term PPAR gamma (PPARγ) as used herein refers to Peroxisome proliferator-activated receptor gamma. PPAR-γ (or PPARG), also known as the glitazone receptor, or NR1C3 (nuclear receptor subfamily 1, group C, member 3) is a type II nuclear receptor that in humans is encoded by the PPARG gene.

The term “sample” as used herein refers to a biological sample obtained for the purpose of evaluation in vitro. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

The term “sarcomere” as used herein refers to a unit of muscle tissue (e.g., heart muscle tissue).

By “specifically binds” is meant a compound or antibody that recognizes and binds a polypeptide of the invention, but which does not substantially recognize and bind other molecules in a sample, for example, a biological sample, which naturally includes a polypeptide of the invention.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with heart disease, neurodegenerative disorders, etc. is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The terms “treat,” treating,” “treatment,” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

The terms “prevent”, “preventing”, “prevention”, “prophylactic treatment” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

A “therapeutically effective amount” is an amount sufficient to effect beneficial or desired results, including clinical results. An effective amount can be administered in one or more administrations.

Any compositions or methods provided herein can be combined with one or more of any of the other compositions and methods provided herein.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1J is a series of photomicrographs and bar charts showing that in vivo-matured PSC-CMs show adult CM morphology. FIG. 1A is a photomicrograph showing α-Actinin (cyan) staining of mESC-CMs matured in vitro for 10 or 60 days (top) and endogenous mouse CMs at postnatal day 1 and 2 months (adult) (bottom). DAPI (blue) was used to counterstain nuclei. FIG. 1B is a photomicrograph showing a 3D image of mESC-CMs matured in the rat heart for 2 months. FIG. 1C is a photomicrograph showing CX43 staining of mESC-CMs matured in the rat heart. FIG. 1D is a photomicrograph showing an adult mouse heart section stained with Rat cTnT (top) and mESC-CMs (GFP+) matured in the rat heart (bottom). Red dotted line indicates mESC-CMs. Inset (bottom right) shows a magnified image of the white box showing well-organized sarcomeres. FIG. 1E is a photomicrograph showing in vivo-matured mESC-CM (GFP+) isolated from the rat heart (top) and adult rat CMs (bottom). FIG. 1F is a bar chart showing average cell length and width of mouse CMs and in vivo-matured mESC-CMs at indicated stages. Data are mean±SD; n=7 per group; *p<0.05; ***p<0.001; ns, not significant (p>0.05). p values were determined using the paired Student t test. FIG. 1G is a photomicrograph showing binucleation % of adult mouse CMs (n=3 mouse hearts) and in vivo-matured mESC-CMs (n=3 rat hearts). FIG. 1H is a photomicrograph showing representative T-tubule images of adult rat CMs (two CMs, left) and in vivo-matured mESC-CM (GFP+) labeled with Di-8-ANNEPS. FIG. 1I is a series of graphs showing fluorescent profiles of adult rat CMs and in vivo-matured mESC-CMs labeled with Di-8-ANNEPS. Top left or right plot corresponds to red- or blue-boxed area in H, respectively. AU, arbitrary units; Bottom plot shows quantification of T-tubule peak intensity (n=40 from 3 cells each). FIG. 1J is a photomicrograph showing transmission electron micrographs of adult rat CM and in vivo-matured mESC-CM. D, day; M, month; p, postnatal day; .H, H band; M, mitochondria; S, sarcomere; SR, sarcoplasmic reticulum; Tt, T-tubule; Z, Z-line. Student's t test and one way-ANOVA were used for statistical analyses.

FIG. 2A-FIG. 2D is a series of graphs showing that in vivo-matured mESC-CMs show adult CM function. FIG. 2A is a graph showing definitions for Ca²⁺ transient analysis. FIG. 2B is a series of graphs showing representative trace and quantification of Ca²⁺ transients, time for peak and baseline 50% and 90% for in vitro-matured mESC-CMs at day 10 (n=13) and 1 month (n=10) and in vivo-matured mESC-CMs at 1 month (n=14). FIG. 2C is a series of graphs showing representative Ca²⁺ transients and sarcomere shortening of endogenous mouse CMs and in vivo-matured mESC-CMs at indicated stages, stimulated at 0.5 Hz with pulse. FIG. 2D is a series of graphs showing quantifications of peak amplitude of Ca²⁺ transients and sarcomere shortening, time to peak and peak 50%, and time to baseline 50% and 90% measured with endogenous mouse CMs at 1 month (n=10) or adult stage (n=7) and within vivo-matured mPSC-CMs at 1 month (n=8-14) or 2 months (n=13). Data are mean±SD; *p<0.05; **p<0.01; ***p<0.001; ****p<0.0001; ns, not significant (p>0.05). p values were determined using the One-way ANOVA (B) or Two-way ANOVA (D) with non-parametric multiple comparison.

FIG. 3A-FIG. 3D is a series of schematics and illustrations regarding single-cell RNA-seq analysis. FIG. 3A is an outline of RNA-seq pipeline for data analysis. FIG. 3B is a heatmap visualization of hierarchically clustered samples showing high (red) and low (blue) expression of 8 in vitro-matured mESC-CMs, 8 in vivo-matured mESC-CMs and 8 adult mouse CMs. FIG. 3C is a dot plot showing PCA of gene expression of in vitro (red), in vivo (blue), and adult (green) CMs. FIG. 3D is a treemap plot of gene ontology (GO) analysis of differentially expressed genes showing superclusters of related terms.

FIG. 4A-FIG. 4D is a series of photomicrographs and graphs showing that in vivo-matured hiPSC-CMs become adult-like CMs. FIG. 4A is a photomicrograph showing hiPSC-CMs (GFP) engrafted in the rat heart. FIG. 4B is a series of high resolution images of adult rat CMs (two CMs) and in vivo-matured hiPSC-CM showing well-organized sarcomeric structure. Boxed regions are enlarged in the bottom. FIG. 4C shows representative sarcomere shortening and Ca²⁺ transients for in vivo-matured hiPSC-CMs compared to adult human CMs. FIG. 4D is a series of graphs showing quantifications of peak amplitude of sarcomere shortening and C²⁺ transients of adult human CMs (n=10) and in vivo-matured hiPSC-CMs (n=12). SS, sarcomere shortening. Data are mean±SD; ns, not significant (p>0.05). p values were determined using the non-parametric Mann-Whitney test.

FIG. SA-FIG. 5G is a series of photomicrographs and a bar chart showing that in vivo-matured ARVC-hiPSC-CMs exhibit human ARVC disease phenotype. FIG. 5A is a series of photomicrogrpahs showing adult WT/ARVC mouse heart sections stained with antibodies against Perilipin (yellow) and cTnT (green) and DAPI. Perilipin is a lipid droplet-associated protein. FIG. 5B is a series of photomicrogrpahs showing adult WT/ARVC mouse heart sections stained with TUNEL (red), showing apoptotic cells. FIG. 5C is a series of photomicrographs showing in vitro-matured GFP-labeled ARVC hiPSC-CMs stained with Perilipin (red), GFP (green), and a-Actinin (cyan) antibody (top) and ARVC-CMs (bottom) stained with antibodies against Perilipin (red), α-Actinin (cyan) and GFP (green). DAPI (blue) was used to counterstain nuclei. FIG. 5D is a series of photomicrogrpahs showing in vivo-matured GFP-labeled ARVC hiPSC-CMs. FIG. 5E is a series of photomicrographs showing TUNEL staining of control hiPSC-CMs and ARVC hiPSC-CMs matured in vivo. FIG. 5F is a series of photomicrogrpahs showing quantification of TUNEL positive CMs. Data are mean±SD; section number=3; hiPSC CMs (n=391), ARVC CMs (n=430); ***p<0.001; p values were determined using the paired Student t test. FIG. 5G is a bar chart showing transmission electron micrographs of human control, ARVD patient CMs, and in vivo-matured ARVC hiPSC-CMs. n=10 Rats. Blue arrows indicate intercalated disc abnormalities (widening of intercellular space).

FIG. 6A-FIG. 6B is a schematic and a series of photomicrographs. FIG. 6A is a schematic for in-vitro differentiation and in vivo maturation of PSC-CPCs/CMs. FIG. 6B is a series of photomicrographs showing rat heart with mESC-CMs incubated for 1 month. RFP and GFP are mESC-CM lineage tracers.

FIG. 7A-FIG. 7E is a series of photomicrographs and a bar chart showing PSC-CMs matured in vivo. FIG. 7A is a photomicrograph showing heart sections showing mESCIsl1-Cre; Rosa-RFP; aMHC-GFP-CMs incubated for 1 week (top) and 1 month (bottom), stained with cTnT antibody (cyan). White arrows indicate maturing mESC-CMs. FIG. 7B is a series of photomicrographs showing mESC-CMs injected at P14 and matured for 1 month in vivo. They remain small in size with disorganized sarcomere structure (top: heart section, bottom: isolated CMs). FIG. 7C is a series of photomicrographs showing in vivo-matured mESC-CMs stained with cTnT antibody that cross-reacts with both mouse and rat. FIG. 7D is a series of photomicrographs showing in vitro-matured mESC-CMs labeled with Di-8-ANNEPS. FIG. 7E is a bar chart showing binucleation % of adult mouse CMs and in vivo-matured hiPSC-CMs. n=3 mice and n=3 rats.

FIG. 8A-FIG. 8F is a series of graphs showing the effect of secreted/membrane-bound factors enriched in early postnatal hearts on CM maturation. FIG. 8A is a gene expression heatmap of 6 extracellular or membrane-bound genes in whole heart samples. All genes are upregulated at postnatal stages. FIG. 8B is a gene expression heatmap of 16 nuclear receptor (NR) genes demonstrating upregulation from embryonic to adult stages. FIG. 8C is an experimental scheme of mESC-CMs treatment with 6 factors (activators and recombinant proteins) for 4, 7 and 14 days. FIG. 8D is a bar chart showing that after 4 days of treatment with 6 factors, only one NR gene (THRa) was significantly upregulated. FIG. 8E is a bar chart showing that after 7 days of treatment with 6 factors, 6 NR genes (THRa, Ar, PPARd, PPARg, Esrra and Cebpa) were significantly upregulated and one (Cebpb) was downregulated. FIG. 8F is a bar chart showing that after 14 days of treatment with 6 factors, 10 out of 16 NRs (THRa, Ar, PPARa, PPARd, PPARg, Rora, Rxra, Esrra, Esr2 and Cebpa) were significantly upregulated and none of the NRs was suppressed. *p<0.05, **p<0.01, ***p<0.001. p values were determined using the paired Student t test.

FIG. 9A-FIG. 9F is a series of graphs showing gene expression analysis and functional characterization of hiPSC-derived neurons in vitro and in vivo. FIG. 9A is a series of photomicrographs showing dissected 2 months old Rats brain. RFP signaling is a lineage tracer of injected hiPSC-NPCs. FIG. 9B is a series of photomicrographs wherein brain slices were stained with DAPI (blue) and MAP2(green). White arrows indicate RFP and GFP overlapping neurons. FIG. 9C is a series of bar graphs showing the relative expression pattern between in vitro and in vivo matured neuron. FIG. 9D is a series of graphs showing current-clamp recordings from hiPSC-derived neurons showing action potential firing at depolarized potentials. Increased action potential firing occurred with increasing current injection in hiPSC-derived neurons in brain slices. FIG. 9E is a series of representative traces showing Na⁺ and K⁺ currents and their I-V curve recorded from hiPSC-derived neurons in cultures and in slices. FIG. 9F is a series of graphs showing spontaneous synaptic events recorded from hiPSC-derived neurons, showing that hiPSC-derived neurons displayed more extensive synaptic activities in slices than in cultures. All events were blocked by CNQX (6-Cyano-7-nitroquinoxaline-2,3-dione, 10 μM) and Bic (Bicuculline, 20 μM). Data are expressed as mean±SEM (*p<0.05, two-tailed t-test). CT, cortex; CB, cerebellum; CA, cornu ammonis; DG, dendate gyms.

### DETAILED DESCRIPTION OF THE INVENTION

The present invention is based, at least in part, upon the development of compositions and methods for treating diseases using adult cells. In particular, the present invention provides for methods of treating disease (e.g., cardiomyopathy) by administering adult cells produced from pluripotent stem cells (PSC). Furthermore, the present invention provides methods of maturing PSC-derived cells into adult cells via incubation of the PSC-derived cells with differentiated adult cells (e.g., the postnatal cellular environment) of the desired cell type. In some embodiments, PSC cells are induced into immature PSC-derived cells in an in vitro setting (e.g., a dish), prior to incubation with the postnatal cellular environment. For example, the PSC-derived cells are incubated within an organ, e.g., a heart, to induce the maturation of the PSC. In some embodiments, the PSC-derived cell (such as cardiac progenitor cells—CPCs) is incubated with a fully differentiated cell, e.g., those from a heart, to induce the immature PSC-derived cells to become adult (e.g., cardiomyocyte) cells. In some embodiments, the immature PSC-derived cell is incubated with differentiated tissue of another species. For example, a human PSC-derived cell is incubated with rodent adult cells, e.g., a rodent heart, to generate human adult heart cells.

The PSC field is intensively focused on heart disease because of its worldwide prevalence causing high morbidity and mortality. In particular, with methodological advances in differentiating PSCs into cardiomyocytes (CMs), current cardiac PSC research is focusing on modeling cardiomyopathy—a leading cause of heart failure resulting from CM gene defects—and PSC-based cell therapy to treat myocardial infarction—a leading cause of human mortality (Kamdar, F., et al. (2015). J Card Fail; Lalit, P.A., et al. (2014). Circ Res 114, 1328-1345). However, cardiomyopathy occurs predominantly in adult stages. Thus, prior to the invention described herein, it was difficult to recapitulate the true disease phenotype and to validate the efficacy of drugs discovered with PSC-derived CMs (PSC-CMs). For this reason, extensive tissue engineering efforts are underway with the goal to mature PSC-CMs in vitro. Similarly, prior to the invention described herein, the beneficial effect of PSC-CM-based myocardial repair was in question, as they did not properly mature in adult environments (Cho, G. S., Fernandez, L., and Kwon, C. (2014). Antioxid Redox Signal 21, 2018-2031).

As described herein, studies demonstrate that electrical and mechanical stimulations promote the structural and functional maturation of PSC-CMs. Substrate properties were also shown to play an important role in their maturation. For instance, myofibril alignment and contractility were significantly enhanced in PSC-CMs grown in micropatterned polyacrylamide. These studies evince the critical role of the microenvironment in PSC-CM maturation.

Pluripotent stem cells (PSCs)—isolated from early embryos (ESCs) or induced from adult cells with reprogramming factors (iPSCs) (Martin, G. R. (1981). Proc Natl Acad Sci USA 78, 7634-7638; Takahashi, K. and Yamanaka, S. (2006) Cell 126, 663-676)—are capable of becoming any type of body cell. It has been a decade since Yamanaka and colleagues found a way to turn adult cells back to pluripotent stem cells (PSCs), named induced PSCs (iPSCs). Thus, PSCs are useful in disease modeling, drug discovery, and regenerative medicine, as well as understanding human development (Fox, I. J., et al. (2014). Science 345, 1247391; Tabar, V., and Studer, L. (2014). Nat Rev Genet 15, 82-92). For this reason, a number of PSCs have been generated from embryos or patients harboring various mutations or diseases for preclinical studies and clinical therapeutics. While some of these studies demonstrated successful differentiation of PSCs into specific cell types with their potential to model and treat a broad spectrum of human diseases, prior to the invention described herein, PSC-derived cells were morphologically and functionally similar to fetal cells, i.e., PSC-derived cells were similar to immature cells and exhibited fetal-like characteristics and remained immature in a dish. This immaturity was a major and common impediment for their application to model and treat adult-onset (i.e., late-onset) disorders such as cardiomyopathy (Mercola, M., Colas, A., and Willems, E. (2013Circ Res 112, 534-548; Tabar, V., and Studer, L. (2014). Nat Rev Genet 15, 82-92).

Prior to the invention described herein, the only method to generate adult CMs from PSCs was via blastocyst injection, but this method is not applicable in humans. Numerous tissue engineering approaches, including electrical, mechanical, and environmental stimulations, were shown to enhance the maturation of PSC-CMs (Chun, Y. W., et al.(2015). Biomaterials 67, 52-64; Kuppusamy, K. T., et al. (2015). Proc Natl Acad Sci USA 112, E2785-2794; Nunes, S. S., et al. (2013). Nat Methods 10, 781-787; Rog-Zielinska, E. A., et al. (2015). Cell Death Differ 22, 1106-1116; Ruan, J. L., et al.(2015). Stem Cells 33, 2148-2157). However, prior to the invention described herein, there were not any reports on the generation of adult-like CMs from human PSCs despite extensive tissue-engineering efforts. This evinces the complexity of CM maturation. Indeed, maturation of cardiomyocytes occurs over a long period of time (>10 years) in humans. Described herein are methods wherein human PSC-derived cardiomyocytes undergo full maturation in about 4-6 weeks (as compared to about 10 years) when introduced into the heart of young animals. Immature PSC-derived cells (e.g., immature cardiomyocytes) are induced from PSC cells in vitro (e.g., in a dish) prior to maturation in the postnatal cellular environment, where they will become adult cells.

Developmentally, the maturation of CMs begins at an early embryonic stage and continues throughout the postnatal stages. PSC-CMs resemble early embryonic CMs in structure, function, and gene expression. Transcriptional analysis revealed that PSC-CMs undergo maturation in culture, but are arrested at late embryonic/neonatal stages. Described in detail below is the leveraging of the potential of the neonatal heart environment to demonstrate that neonatal hearts are capable of maturing PSC-CMs to adult-like CMs. As described herein, the resulting cardiomyocytes are directly used for drug screening, disease modeling, and cell therapy to treat heart muscle diseases.

The PSC-derived cells described herein can be genetically labeled with markers such as fluorescent or bacterial proteins to identify and/or isolate the cells upon maturation. PSC-derived cells can also be identified and isolated using species-specific antibodies when different species are used for the postnatal cellular environment.

One purpose of the methods described herein is to make adult cardiac muscle cells from PSCs. This technology allows for: 1.) generating adult cardiac muscle cells from human pluripotent stem cells; 2.) using the resulting human cardiac muscle cells for drug testing, drug screening, and regeneration therapy to treat heart diseases (can be immediately used for preclinical trials with patients' own cells); and 3.) using animals as a microenvironment to generate adult cell types. In addition to heart disease (regarded as the number one killer worldwide), a number of diseases such as Alzheimer's disease and Parkinson's disease manifest in adult stages. However, prior to the invention described herein, there were no reliable methods to generate adult cell types from patient-specific PSCs, e.g., to treat such adult-onset diseases. Described herein are compositions and methods to treat heart diseases, Parkinson's disease, Alzheimer's disease, and other adult-onset diseases.

Pluripotent stem cells provide unprecedented opportunities for disease modeling and personalized medicine. Described herein are PSC-derived cardiomyocytes (PSC-CMs) that undergo full maturation when introduced into early postnatal hearts, e.g., within 2 weeks from birth. As described in detail below, when incubated in neonatal hearts, PSC-CMs became similar to adult CMs in morphology and function within a month. The similarity was further supported by single-cell RNA-sequencing analysis, also known as whole transcriptome shotgun sequencing (WTSS), which uses next-generation sequencing (NGS to reveal the presence and quantity of RNA in a biological sample at a given moment in time. See, e.g., Wang et al., 2009 Nat Rev Genet, 10(1): 57-63, incorporated herein by reference.

Specifically, in vivo-incubated PSC-CMs were similar to adult CMs in size and sarcomere length and formed structures that are absent in immature CMs including t-tubules. Functionally, PSC-CMs displayed sarcomere shortening and calcium transients that were similar to adult CMs. Moreover, the neonatal system allowed patient-derived PSC-CMs to reveal the disease phenotype of arrhythomogenic right ventricular cardiomyopathy, which predominantly manifests in adults. As described in detail below, the neonatal rodent heart functions as a simple yet powerful tool to generate adult-like CMs from PSCs. Thus, the compositions and methods described herein provide additional information on the human CM maturation and pathogenesis of human CMs and open a new avenue for PSC-based modeling and treatment of adult heart diseases.

### Stem Cells

Embryonic stem cells (ES cells) are pluripotent stem cells derived from the inner cell mass of a blastocyst, an early-stage preimplantation embryo. Human embryos reach the blastocyst stage within 4-5 days post fertilization, at which time they consist of 50-150 cells. Isolating the embryoblast or inner cell mass (ICM) results in destruction of the blastocyst. Embryonic stem cells, derived from the blastocyst stage early mammalian embryos, are distinguished by their ability to differentiate into any cell type and by their ability to propagate. Embryonic stem cell properties include having a normal karyotype, maintaining high telomerase activity, and exhibiting remarkable long-term proliferative potential.

Embryonic stem cells of the inner cell mass are pluripotent, that is, they are able to differentiate to generate primitive ectoderm, which ultimately differentiates during gastrulation into all derivatives of the three primary germ layers: ectoderm, endoderm, and mesoderm. These include each of the more than 220 cell types in the adult body. Pluripotency distinguishes embryonic stem cells from adult stem cells found in adults. While embryonic stem cells can generate all cell types in the body, adult stem cells are multipotent and can produce only a limited number of cell types. Harnessing the pluripotent differentiation potential of embryonic stem cells in vitro provide a means of deriving cell or tissue types virtually to order. This would provide a radical new treatment approach to a wide variety of conditions where age, disease, or trauma has led to tissue damage or dysfunction.

Additionally, under defined conditions, embryonic stem cells are capable of propagating themselves indefinitely in an undifferentiated state and have the capacity when provided with the appropriate signals to differentiate, presumably via the formation of precursor cells, to almost all mature cell phenotypes. This allows embryonic stem cells to be employed as useful tools for both research and regenerative medicine, because they produce limitless numbers of themselves for continued research or clinical use. Because of their plasticity and potentially unlimited capacity for self-renewal, embryonic stem cell therapies are used for regenerative medicine and tissue replacement after injury or disease.

Diseases that could potentially be treated by pluripotent stem cells include a number of blood and immune-system related genetic diseases, cancers, and disorders, e.g., juvenile diabetes, Parkinson's, blindness, and spinal cord injuries. There is a technical problem of graft-versus-host disease associated with allogeneic stem cell transplantation. However, the problems associated with histocompatibility may be solved using autologous donor adult stem cells or therapeutic cloning. The therapeutic cloning performed by a method called somatic cell nuclear transfer (SCNT) may be advantageous against mitochondrial DNA (mtDNA) mutated diseases.

Induced pluripotent stem cells, commonly abbreviated as iPS cells or iPSCs are a type of pluripotent stem cell artificially derived from a non-pluripotent cell, typically an adult somatic cell, by inducing a “forced” expression of certain genes and transcription factors. These transcription factors play a key role in determining the state of these cells and also highlight the fact that these somatic cells do preserve the same genetic information as early embryonic cells. The ability to induce cells into a pluripotent state was initially pioneered using mouse fibroblasts and four transcription factors, Oct4, Sox2, Klf4 and c-Myc;—called reprogramming. The successful induction of human iPSCs derived from human dermal fibroblasts has been performed using methods similar to those used for the induction of mouse cells. These induced cells exhibit similar traits to those of embryonic stem cells (ESCs) but do not require the use of embryos. Some of the similarities between ESCs and iPSCs include pluripotency, morphology, self-renewal ability, a trait that implies that they can divide and replicate indefinitely, and gene expression. Cardiac progenitor cells (or CPCs) are one type of pluripotent stem cell.

Current research focuses on differentiating ES into a variety of cell types for eventual use as cell replacement therapies (CRTs). Some of the cell types that have or are being developed include cardiomyocytes (CM), neurons, hepatocytes, bone marrow cells, islet cells and endothelial cells. Besides becoming an important alternative to organ transplants, ES are also being used in field of toxicology and as cellular screens to uncover new chemical entities (NCEs) that can be developed as small molecule drugs. Studies have shown that cardiomyocytes derived from ES are validated in in vitro models to test drug responses and predict toxicity profiles.

Adult stem cells, also called somatic stem cells, are stem cells which maintain and repair the tissue in which they are found. They can be found in children, as well as adults. Pluripotent adult stem cells are rare and generally small in number, but they can be found in umbilical cord blood and other tissues. Bone marrow is a rich source of adult stem cells, which have been used in treating several conditions including spinal cord injury, liver cirrhosis, chronic limb ischemia, and endstage heart failure. The quantity of bone marrow stem cells declines with age and is greater in males than females during reproductive years. Much adult stem cell research has aimed to characterize their potency and self-renewal capabilities. DNA damage accumulates with age in both stem cells and the cells that comprise the stem cell environment. This accumulation is considered to be responsible, at least in part, for increasing stem cell dysfunction with aging (see DNA damage theory of aging).

In adult organisms, stem cells and progenitor cells act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. There are three known accessible sources of autologous adult stem cells in humans: 1. Bone marrow, which requires extraction by harvesting, that is, drilling into bone (typically the femur or iliac crest). 2. Adipose tissue (lipid cells), which requires extraction by liposuction. 3. Blood, which requires extraction through apheresis, wherein blood is drawn from the donor (similar to a blood donation), and passed through a machine that extracts the stem cells and returns other portions of the blood to the donor. Stem cells can also be taken from umbilical cord blood just after birth. Of all stem cell types, autologous harvesting involves the least risk. By definition, autologous cells are obtained from one's own body.

Most adult stem cells are lineage-restricted (multipotent) and are generally referred to by their tissue origin (mesenchymal stem cell, adipose-derived stem cell, endothelial stem cell, dental pulp stem cell, etc.). Adult stem cell treatments have been successfully used for many years to treat leukemia and related bone/blood cancers through bone marrow transplants. Adult stem cells are also used in veterinary medicine to treat tendon and ligament injuries in horses. In instances where adult stem cells are obtained from the intended recipient (an autograft), the risk of rejection is essentially non-existent.

### Cardiomyocytes

Cardiac muscle cells or cardiomyocytes (also known as myocardiocytes or cardiac myocytes) are the muscle cells (myocytes) that make up the cardiac muscle. Each myocardial cell contains myofibrils, which are specialized organelles consisting of long chains of sarcomeres, the fundamental contractile units of muscle cells. Cardiomyocytes show striations similar to those on skeletal muscle cells, but unlike multinucleated skeletal cells, they contain only one nucleus. Cardiomyocytes have a high mitochondrial density, which allows them to produce adenosine triphosphate (ATP) quickly, making them highly resistant to fatigue.

There are two types of cells within the heart: the cardiomyocytes and the cardiac pacemaker cells. Cardiomyocytes make up the atria (the chambers in which blood enters the heart) and the ventricles (the chambers where blood is collected and pumped out of the heart). These cells must be able to shorten and lengthen their fibers and the fibers must be flexible enough to stretch. These functions are critical to the proper form during the beating of the heart.

Cardiac pacemaker cells carry the impulses that are responsible for the beating of the heart. They are distributed throughout the heart and are responsible for several functions. First, they are responsible for being able to spontaneously generate and send out electrical impulses. They also must be able to receive and respond to electrical impulses from the brain. Lastly, they must be able to transfer electrical impulses from cell to cell.

All of these cells are connected by cellular bridges. Porous junctions called intercalated discs form junctions between the cells. They permit sodium, potassium and calcium to easily diffuse from cell to cell. This makes it easier for depolarization and repolarization in the myocardium. Because of these junctions and bridges the heart muscle is able to act as a single coordinated unit.

Cardiac action potential consists of two cycles, a rest phase and an active phase. These two phases are commonly understood as systole and diastole. The rest phase is considered polarized. The resting potential during this phase of the beat separates the ions such as sodium, potassium and calcium. Myocardial cells possess the property of automaticity or spontaneous depolarization. This is the direct result of a membrane which allows sodium ions to slowly enter the cell until the threshold is reached for depolarization. Calcium ions follow and extend the depolarization even further. Once calcium stops moving inward, potassium ions move out slowly to produce repolarization. The very slow repolarization of the CMC membrane is responsible for the long refractory period.

Myocardial infarction, commonly known as a heart attack, occurs when the heart's supplementary blood vessels are obstructed by an unstable build-up of white blood cells, cholesterol, and fat. With no blood flow, the cells die, causing whole portions of cardiac tissue to die. Once these tissues are lost, they cannot be replaced, thus causing permanent damage.

Humans are born with a set number of heart muscle cells, or cardiomyocytes, which increase in size as our heart grows larger during childhood development. Recent evidence suggests that cardiomyocytes are actually slowly turned over as we age, but that less than 50% of the cardiomyocytes we are born with are replaced during a normal life span. The growth of individual cardiomyocytes not only occurs during normal heart development, it also occurs in response to extensive exercise (athletic heart syndrome), heart disease, or heart muscle injury such as after a myocardial infarction. A healthy adult cardiomyocyte has a cylindrical shape that is approximately 100 μm long and 10-25 μm in diameter. Cardiomyocyte hypertrophy occurs through sarcomerogenesis, the creation of new sarcomere units in the cell. During heart volume overload, cardiomyocytes grow through eccentric hypertrophy. The cardiomyocytes extend lengthwise but have the same diameter, resulting in ventricular dilation. During heart pressure overload, cardiomyocytes grow through concentric hypertrophy. The cardiomyocytes grow larger in diameter but have the same length, resulting in heart wall thickening.

### Heart Disease

Cardiovascular disease (CVD) is a class of diseases that involve the heart or blood vessels. Cardiovascular disease includes coronary artery diseases (CAD) such as angina and myocardial infarction (commonly known as a heart attack). Other CVDs are stroke, hypertensive heart disease, rheumatic heart disease, cardiomyopathy, atrial fibrillation, congenital heart disease, endocarditis, aortic aneurysms, peripheral artery disease and venous thrombosis.

A number of methods exist for diagnosing heart disease. Screening ECGs (Electrocardiogram—either at rest or with exercise) are one way to detect heart disease. Additionally echocardiography, myocardial perfusion imaging, and cardiac stress testing is not recommended in those at low risk who do not have symptoms. Some biomarkers may add to conventional cardiovascular risk factors in predicting the risk of future cardiovascular disease.

The underlying mechanisms vary depending on the disease in question. Coronary artery disease, stroke, and peripheral artery disease involve atherosclerosis. This may be caused by high blood pressure, smoking, diabetes, lack of exercise, obesity, high blood cholesterol, poor diet, and excessive alcohol consumption, among others. High blood pressure results in 13% of CVD deaths, while tobacco results in 9%, diabetes 6%, lack of exercise 6% and obesity 5%. Rheumatic heart disease may follow untreated strep throat.

There are many cardiovascular diseases involving the blood vessels. They are known as vascular diseases: Coronary artery disease (also known as coronary heart disease and ischemic heart disease), Peripheral arterial disease—disease of blood vessels that supply blood to the arms and legs, Cerebrovascular disease—disease of blood vessels that supply blood to the brain (includes stroke), Renal artery stenosis and Aortic aneurysm. There are also many cardiovascular diseases that involve the heart including but not limited to:

Cardiomyopathy—diseases of cardiac muscle, Hypertensive heart disease—diseases of the heart secondary to high blood pressure or hypertension, Heart failure, Pulmonary heart disease—a failure at the right side of the heart with respiratory system involvement, Cardiac dysrhythmias—abnormalities of heart rhythm, Inflammatory heart disease,

Endocarditis—inflammation of the inner layer of the heart, the endocardium. (The structures most commonly involved are the heart valves.) Inflammatory cardiomegaly, Myocarditis—inflammation of the myocardium, the muscular part of the heart, Valvular heart disease, Congenital heart disease—heart structure malformations existing at birth, Rheumatic heart disease—heart muscles and valves damage due to rheumatic fever caused by Streptococcus pyogenes a group A streptococcal infection.

There are several risk factors for heart diseases: age, gender, tobacco use, physical inactivity, excessive alcohol consumption, unhealthy diet, obesity, family history of cardiovascular disease, raised blood pressure (hypertension), raised blood sugar (diabetes mellitus), raised blood cholesterol (hyperlipidemia), psychosocial factors, poverty and low educational status, and air pollution. While the individual contribution of each risk factor varies between different communities or ethnic groups the overall contribution of these risk factors is very consistent. Some of these risk factors, such as age, gender or family history, are immutable; however, many important cardiovascular risk factors are modifiable by lifestyle change, social change, drug treatment and prevention of hypertension, hyperlipidemia, and diabetes.

### Cardiomyopathy

Cardiomyopathy (literally “heart muscle disease”) is the measurable deterioration for any reason of the ability of the myocardium (the heart muscle) to contract, usually leading to heart failure. Common symptoms include dyspnea (breathlessness) and peripheral edema (swelling of the legs). Those with cardiomyopathy are often at risk of dangerous forms of irregular heart rate and sudden cardiac death. The most common form of cardiomyopathy is dilated cardiomyopathy. Although the term “cardiomyopathy” could theoretically apply to almost any disease affecting the heart, it is usually reserved for “severe myocardial disease leading to heart failure”. Cardiomyopathy and myocarditis resulted in 443,000 deaths in 2013, up from 294,000 in 1990.

Cardiomyopathies are either confined to the heart or are part of a generalized disorder, both often leading to death or progressive heart failure. Other diseases that cause heart muscle dysfunction are excluded, such as coronary artery disease, hypertension, or abnormalities of the heart valves.

Earlier, simpler, categories such as intrinsic, (defined as weakness of the heart muscle without an identifiable external cause), and extrinsic, (where the primary pathology arose outside the myocardium itself), became more difficult to sustain. For example, as more external causes were recognized, the intrinsic category became smaller. Alcoholism, for example, has been identified as a cause of dilated cardiomyopathy, as has drug toxicity, and certain infections (including Hepatitis C). On the other hand, molecular biology and genetics have given rise to the recognition of various genetic causes, increasing the intrinsic category. For example, mutations in the cardiac desmosomal genes as well as in the DES gene may cause arrhythmogenic right ventricular cardiomyopathy (ARVC).

At the same time, a more clinical categorization of cardiomyopathy as ‘hypertrophied’, ‘dilated’, or ‘restrictive’, became difficult to maintain when it became apparent that some of the conditions could fulfill more than one of those three categories at any particular stage of their development. The current American Heart Association definition divides cardiomyopathies into primary, which affect the heart alone, and secondary, which are the result of illness affecting other parts of the body. These categories are further broken down into subgroups which incorporate new genetic and molecular biology knowledge.

Cardiomyopathies can be classified using different criteria. Structural categories of cardiomyopathy include but are not limited to: Primary/intrinsic cardiomyopathies, Genetic Hypertrophic cardiomyopathy, Arrhythmogenic right ventricular cardiomyopathy (ARVC), LV non-compaction, Ion Channelopathies, Dilated cardiomyopathy (DCM), Restrictive cardiomyopathy (RCM), Aquired Cardiommyopathy, Stress Cardiomyopathy, Myocarditis, and Ischemic cardiomyopathy. Secondary/extrinsic cardiomyopathies include but are not limited to: Metabolic/storage disease, Fabry's disease, hemochromatosis, Endomyocardial fibrosis, Hypereosinophilic syndrome, diabetes mellitus, hyperthyroidism, acromegaly, Noonan syndrome, muscular dystrophy, Friedreich's ataxia, and Obesity-associated cardiomyopathy.

Symptoms may include shortness of breath after physical exertion, fatigue, and swelling of the feet, legs, or abdomen. Additionally, arrhythmias and chest pain may be present. The pathophysiology of cardiomyopathies is better understood at the cellular level with advances in molecular techniques. Mutant proteins can disturb cardiac function in the contractile apparatus (or mechanosensitive complexes). Cardiomyocyte alterations and their persistent responses at the cellular level cause changes that are correlated with sudden cardiac death and other cardiac problems. A number of methods exist for detecting the presence of cardiomyopathy in a patient. Among the diagnostic procedures done to determine a cardiomyopathy are: Physical exam, Family history, Blood test, EKG, Echocardiogram, Stress test, and Genetic testing.

Treatment may include suggestion of lifestyle changes to better manage the condition. Treatment depends on the type of cardiomyopathy and condition of disease, but may include medication (conservative treatment) or iatrogenic/implanted pacemakers for slow heart rates, defibrillators for those prone to fatal heart rhythms, ventricular assist devices (VADs) for severe heart failure, or ablation for recurring dysrhythmias that cannot be eliminated by medication or mechanical cardioversion. The goal of treatment is often symptom relief, and some patients may eventually require a heart transplant.

### Peroxisome Proliferator-Activated Receptor Gamma (PPARγ or PPARG)

PPARG is mainly present in adipose tissue, colon and macrophages. Two isoforms of PPARG are detected in the human and in the mouse: PPAR-γ1 (found in nearly all tissues except muscle) and PPAR-γ2 (mostly found in adipose tissue and the intestine). PPARG regulates fatty acid storage and glucose metabolism. This gene encodes a member of the peroxisome proliferator-activated receptor (PPAR) subfamily of nuclear receptors. PPARs form heterodimers with retinoid X receptors (RXRs) and these heterodimers regulate transcription of various genes. Three subtypes of PPARs are known: PPAR-alpha, PPAR-delta, and PPAR-gamma. The protein encoded by this gene is PPAR-gamma and is a regulator of adipocyte differentiation. Alternatively spliced transcript variants that encode different isoforms have been described. Many naturally occurring agents directly bind with and activate PPAR gamma. These agents include various polyunsaturated fatty acids like arachidonic acid and arachidonic acid metabolites such as certain members of the 5-Hydroxyicosatetraenoic acid and 5-oxo-eicosatetraenoic acid family, e.g. 5-oxo-15(S)-HETE and 5-oxo-ETE or 15-Hydroxyicosatetraenoic acid family including 15(S)-HETE, 15(R)-HETE, and 15(S)-HpETE. PPAR-gamma has been implicated in the pathology of numerous diseases including obesity, diabetes, atherosclerosis, and cancer. PPAR-gamma agonists have been used in the treatment of hyperlipidaemia and hyperglycemia. PPAR-gamma decreases the inflammatory response of many cardiovascular cells, particularly endothelial cells. PPAR-gamma activates the PON1 gene, increasing synthesis and release of paraoxonase 1 from the liver, reducing atherosclerosis.

### Sarcomere

A sarcomere is the basic unit of striated muscle tissue. Skeletal muscles are composed of tubular muscle cells (myocytes called muscle fibers) which are formed in a process known as myogenesis. Muscle fibers are composed of tubular myofibrils. Myofibrils are composed of repeating sections of sarcomeres, which appear under the microscope as dark and light bands. Sarcomeres are composed of long, fibrous proteins as filaments that slide past each other when a muscle contracts or relaxes. Two of the important proteins are myosin, which forms the thick filament, and actin, which forms the thin filament. Myosin has a long, fibrous tail and a globular head, which binds to actin. The myosin head also binds to ATP, which is the source of energy for muscle movement. Myosin can only bind to actin when the binding sites on actin are exposed by calcium ions. Actin molecules are bound to the Z line, which forms the borders of the sarcomere. Other bands appear when the sarcomere is relaxed.

### Neurodegenerative Disorders

Neurodegeneration is the umbrella term for the progressive loss of structure or function of neurons, including death of neurons. Many neurodegenerative diseases including amyotrophic lateral sclerosis, Parkinson's, Alzheimer's, and Huntington's occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

Many neurodegenerative diseases are caused by genetic mutations, most of which are located in completely unrelated genes. In many of the different diseases, the mutated gene has a common feature: a repeat of the CAG nucleotide triplet. CAG encodes for the amino acid glutamine. A repeat of CAG results in a polyglutamine (polyQ) tract. Diseases showing this are known as polyglutamine diseases. Nine inherited neurodegenerative diseases are caused by the expansion of the CAG trinucleotide and polyQ tract. Two examples are Huntington's disease and spinocerebellar ataxias.

Several neurodegenerative diseases are classified as proteopathies as they are associated with the aggregation of misfolded proteins. One such protein is alpha-synuclein which can aggregate to form insoluble fibrils in pathological conditions characterized by Lewy bodies, such as Parkinson's disease, dementia with Lewy bodies, and multiple system atrophy. Alpha-synuclein is the primary structural component of Lewy body fibrils. In addition, an alpha-synuclein fragment, known as the non-Abeta component (NAC), is found in amyloid plaques in Alzheimer's disease. Another protein is tau; when hyperphosphorylated, tau protein is the main component of neurofibrillary tangles in Alzheimer's disease. Also, beta amyloid is the major component of senile plaques in Alzheimer's disease.

Parkinson's disease (PD) and Huntington's disease are both late-onset and associated with the accumulation of intracellular toxic proteins. Diseases caused by the aggregation of proteins are known as proteinopathies, and they are primarily caused by aggregates in the following structures: cytosol (e.g. Parkinson's & Huntington's); nucleus (e.g. Spinocerebellar ataxia type 1 (SCA1)); endoplasmic reticulum (ER), (as seen with neuroserpin mutations that cause familial encephalopathy with neuroserpin inclusion bodies); and extracellularly excreted proteins—amyloid-β in Alzheimer's disease.

Alzheimer's disease is characterised by loss of neurons and synapses in the cerebral cortex and certain subcortical regions. This loss results in gross atrophy of the affected regions, including degeneration in the temporal lobe and parietal lobe, and parts of the frontal cortex and cingulate gyrus Alzheimer's disease has been hypothesized to be a protein misfolding disease (proteopathy), caused by accumulation of abnormally folded A-beta and tau proteins in the brain. Plaques are made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP is critical to neuron growth, survival and post-injury repair. In Alzheimer's disease, an unknown process causes APP to be divided into smaller fragments by enzymes through proteolysis. One of these fragments gives rise to fibrils of beta-amyloid, which form clumps that deposit outside neurons in dense formations known as senile plaques.

Alzheimer's disease is diagnosed through a complete medical assessment. There is no one clinical test that can determine whether a person has Alzheimer's. Usually several tests are performed to rule out any other cause of dementia. The only definitive method of diagnosis is examination of brain tissue obtained from a biopsy or autopsy. Tests (such as blood tests and brain imaging) are used to rule out other causes of dementia-like symptoms. Laboratory tests and screening include: complete blood cell count; electrolyte panel; screening metabolic panel; thyroid gland function tests; vitamin B-12 folate levels; tests for syphilis and, depending on history, for human immunodeficiency antibodies; urinalysis; electrocardiogram (ECG); chest X-ray; computerized tomography (CT) head scan; and an electroencephalogram (EEG). A lumbar puncture may also be informative in the overall diagnosis.

Parkinson's disease manifests as bradykinesia, rigidity, resting tremor and posture instability. Parkinson's disease is a degenerative disorder of the central nervous system. It results from the death of dopamine-generating cells in the substantia nigra, a region of the midbrain; the cause of cell-death is unknown.

The mechanism by which the brain cells in Parkinson's are lost may consist of an abnormal accumulation of the protein alpha-synuclein bound to ubiquitin in the damaged cells. The alpha-synuclein-ubiquitin complex cannot be directed to the proteosome. This protein accumulation forms proteinaceous cytoplasmic inclusions called Lewy bodies. The latest research on pathogenesis of disease has shown that the death of dopaminergic neurons by alpha-synuclein is due to a defect in the machinery that transports proteins between two major cellular organelles—the endoplasmic reticulum (ER) and the Golgi apparatus. Certain proteins like Rab1 may reverse this defect caused by alpha-synuclein in animal models.

Recent research suggests that impaired axonal transport of alpha-synuclein leads to its accumulation in the Lewy bodies. Experiments have revealed reduced transport rates of both wild-type and two familial Parkinson's disease-associated mutant alpha-synucleins through axons of cultured neurons. Membrane damage by alpha-synuclein could be another Parkinson's disease mechanism. Susceptibility genes including a-synuclein, leucine-rich repeat kinase 2 (LRRK-2), and glucocerebrosidase (GBA) have shown that genetic predisposition is another important causal factor.

There are no standard diagnostic tests for Parkinson's disease (PD); diagnosis rests on the clinical information provided by the patient and the findings of a neurological exam. Researchers are working to develop an accurate “biological marker,” such as a blood test or an imaging scan. Imaging tests—such as MRI, ultrasound of the brain, SPECT and PET scans—may also be used to help rule out other disorders. The best objective testing for PD consists of specialized brain scanning techniques that can measure the dopamine system and brain metabolism.

Huntington's Disease causes astrogliosis and loss of medium spiny neurons. Areas of the brain are affected according to their structure and the types of neurons they contain, reducing in size as they cumulatively lose cells. The areas affected are mainly in the striatum, but also the frontal and temporal cortices. The striatum's subthalamic nuclei send control signals to the globus pallidus, which initiates and modulates motion. The weaker signals from subthalamic nuclei thus cause reduced initiation and modulation of movement, resulting in the characteristic movements of the disorder. Mutant Huntingtin is an aggregate-prone protein. During the cells' natural clearance process, these proteins are retrogradely transported to the cell body for destruction by lysosomes. It is a possibility that these mutant protein aggregates damage the retrograde transport of important cargoes such as BDNF by damaging molecular motors as well as microtubules.

Amyotrophic lateral sclerosis (ALS or Lou Gehrig's Disease) is a disease in which motor neurons are selectively targeted for degeneration. In 1993, missense mutations in the gene encoding the antioxidant enzyme Cu/Zn superoxide dismutase 1 (SOD1) were discovered in subsets of patients with familial ALS. This discovery led researchers to focus on unlocking the mechanisms for SOD1-mediated diseases. However, the pathogenic mechanism underlying SOD1 mutant toxicity has yet to be resolved. More recently, TDP-43 and FUS protein aggregates have been implicated in some cases of the disease, and a mutation in chromosome 9 (C9orf72) is thought to be the most common known cause of sporadic ALS. In vitro evidence suggests that the primary cellular sites where SOD1 mutations act are located on astrocytes. Astrocytes then cause the toxic effects on the motor neurons. The specific mechanism of toxicity still needs to be investigated, but the findings implicate cells other than neuron cells in neurodegeneration.

The greatest risk factor for neurodegenerative diseases is aging. Mitochondrial DNA mutations as well as oxidative stress both contribute to aging. Many of these diseases are late-onset, meaning there is some factor that changes as a person ages for each disease. One constant factor is that in each disease, neurons gradually lose function as the disease progresses with age.

Generation of Adult Cells from Human PSCs

Prior to the invention described herein, generation of adult CMs from hPSCs has remained intractable. This suggests substantial complexity to the signaling and stimuli needed for CM maturation that normally takes place over a decade in humans. The results described herein demonstrate that hPSC-CMs can mature to adult-like CMs in one month when incubated in rat neonatal hearts. The neonatal system is expected to serve as a tool to generate mature CMs from hPSCs in an accelerated manner

A number of studies have demonstrated that PSC-CMs develop more adult-like phenotype over time in vitro (Robertson et al., 2013 Stem Cells, 31: 829-837; Yang et al., 2014 Circ Res, 114: 511-523). While the precise status of their maturation remains to be determined, a multi-stage, genome-wide analysis indicated that early or late PSC-CMs in culture resemble early embryonic or late embryonic/neonatal CMs, respectively (Uosaki et al., 2015 Cell reports, 13: 1705-1716). The absence of the extensive T-tubule network also supports their immature nature (Kane et al., 2015 Frontiers in cell and developmental biology, 3:59; Knollmann B C, 2013 Circ Res, 112: 969-976). The in vivo-incubated PSC-CMs, however, displayed features similar to adult CMs in morphology, function, and gene expression. For example, T-tubules were formed with a regular pattern and invagination as adult CMs, and this is consistent with their adult-like calcium transients and sarcomere shortening. Moreover, in vivo-incubated ARVC hiPSC-CMs showed disease phenotypes that appear in adults. This suggests that the in vivo-matured PSCs is highly analogous to adult CMs and that this system can be used to study and model human CM development and late-onset CM-autonomous diseases in vivo and in vitro.

Early postnatal hearts contain endogenous CPCs and immature CMs, and it was identified that exogenously introduced PSC-CPCs/CMs can give rise to mature CMs in the hearts. This finding suggests that early hearts provide the environmental cues necessary to guide PSC-CPCs/CMs to becoming adult-like CMs. The cues might in part come from extracellular factors enriched in postnatal hearts, as they were able to promote the molecular maturation of PSC-CMs in vitro. Curiously, the environmental potential to mature PSC-CMs appears to be lost in adult hearts (Shiba et al., 2012 Nature, 489: 322-325), suggesting the presence of a critical time window required for PSC-CM maturation. It will be of great importance to investigate temporal factors present in early postnatal hearts that mediate the process.

It is worth noting that hPSC-CMs mature into adult-like CMs after a month of incubation in rodent hearts. This suggests that the machinery needed for CM maturation might be conserved in rodents and humans. In fact, although rodents have a shorter lifespan, comparative transcriptional analyses revealed that genes involved in mouse CM maturation are similarly regulated during human heart maturation (Uosaki H and Taguchi Y H, 2016 Genomics, proteomics & bioinformatics, 14: 207-215). Next, it is examined whether hPSC-CMs can also be matured in larger animal models. This approach may be extended for generating other types of adult cells prone to disease, such as skeletal muscle cells, pancreatic cells, renal cells etc., from hiPSCs, which would allow the examination and modeling of adult-onset human diseases.

This invention is further illustrated by the following examples which should not be construed as limiting. The contents of all references, patents, and published patent applications cited throughout this application, as well as the figures, are incorporated herein by reference.

### EXAMPLES

### Example 1: Materials and Methods

### Animals

The animals were randomly allocated to experimental groups and both male and female pups were used for cell delivery. No inclusion or exclusion parameters were used for animal experiments. There was no blinding to the group allocation during the experiment.

### Cell Culture, Differentiation, and Delivery

mESCs and hESC/iPSC were obtained, maintained and differentiated as described (Cheng, P., et al. (2013) Development 140, 2587-2596; Uosaki, H., et al. (2012). PLoS One). For CPC purification, cells were dissociated at day 7 and resuspended in PBS containing 0.1% FBS, 20 mM Hepes and 1 mM EDTA. RFP+CPCs were isolated with SH800 sorter (Sony Biotechnology, Japan). For cell delivery, RNU Rats (Charles River Laboratories) were used as host animals. Postnatal rats were anesthetized by cooling on an ice bed, and a hole was made between 4th and 5th rib. Before injection, cells were mixed with IMDM and matrigel at 60:1 ratio (Laflamme, M. A., et al. (2007). Nat Biotechnol 25, 1015-1024) and injected with Eppendorf FemtoJet® Microinjector (10 μl/injection). To test mycoplasma contamination, we used MycoProbe® Mycoplasma Detection Kit (R&D systems, Catalog #CULoo1B).

TABLE 4
Cell sources

| | Species | Source | Identification |
|---|---|---|---|
| mESC-CMs | Mouse | (Uosaki, 2012 #26) | RFP/GFP expression |
| 1M/2M/Adult mouse CMs | Mouse | C57BL/6 mouse | cTNT Ab/morphology |
| ARVC mouse CMs | Mouse | (Chelko, 2016 #7) | cTNT Ab/morphology |
| 1M/2M/Adult rat CMs | Rat | RNU Rat (Charles River) | cTNT Ab/morphology |
| hiPSC/CMs | Human | (Takahashi, 2006 #8) | GFP |
| Adult human CMs | Human | (Basso, 2006 #9) | Morphology |
| Human ARVD iPSC CMs | Human | (Kim, 2013 #6) | GFP |
| Adult human ARVD CMs | Human | (Basso, 2006 #9) | cTNT Ab/morphology |

### Immunohistochemistry and T-Tubule Staining

For immunohistochemistry, cultured cells and dissected hearts were fixed in 4% paraformaldehyde, blocked for 1 hour with 1% BSA, and incubated overnight with the following primary antibodies: α-Actinin (Sigma A7732), RFP (Clontech Laboratories 5f8), GFP (Life Technologies A11122, A10262), Perilipin (Cell Signaling 9349), cTnT (Thermo Scientific MS 295-P1), Rat and human specific cTnT (Abcam ab45932), Connexin 43 (Sigma, C6219), and human mitochondria (Abcam ab92824). Alexa fluor secondary antibodies (488, 564, 648, Life Technologies) were used for secondary detection. For fluorescent t-tubule staining, isolated mESC-CMs were incubated in 10 μM Di-8-ANEPPS (Invitrogen) for 10 min at room temperature, then washed for 10 min with phosphate-buffered saline (PBS) for imaging.

### Whole-Organ Optical Clearing and Imaging

To visualize the extent of CM differentiation and incorporation, the heart of CPC-injected mice was perfused with ice-cold saline followed by 4% PFA in PBS and post-fixed overnight. The heart was subsequently subjected to Scale CUBIC-1 tissue clearing solution (Susaki, E. A., et al. (2014). Cell 157, 726-739). at 37° C. for 7 days with mild shaking. The solution was exchanged with fresh reagents twice. The optically transparent heart was then mounted in the same solution and imaged using a Zeiss LSM 510 or 710 laser scanning confocal microscope with a 10× 0.3 NA or 20× 0.5 NA objective. 3D renderings of the Z-stack images were made using Imaris (Bitplane).

### Measurement of Calcium Transients and Sarcomere Shortening

Adult CMs were freshly isolated from mice or rats. Hearts were quickly removed from the chest after euthanasia and the aorta was perfused with enzymatic perfusion solution as described in (Bassani R A and Bers D M, 1994 Journal of molecular and cellular cardiology, 26: 1335-1347). The isolated CMs were incubated with 1 mM of the ratiometric Ca2+ indicator dye Fura-2AM (Invitrogen, Molecular Probes) containing 1 mM Ca²⁺ 1× Tyrode solution, and then cells were placed in a perfusion chamber and stimulated at 0.5 Hz with pulses. Changes in sarcomere length and whole cell Ca2+ transients were recorded on an inverted fluorescence microscope (Nikon, TE2000) using IonOptix (Myocam®) software. To measure Ca2+ transients with embryonic and postnatal CMs, hearts were minced and enzymatically dissociated with collagenase and trypsin. Cells were then seeded on Gelatin or Laminin coated cover glasses in 10% serum supplemented SFD medium and analyzed the following day. To measure Calcium Transients and Sarcomere Shortening, mouse (C57BL/6, Jackson laboratory) and RNU Rats (Charles River Laboratories) were used. Differentiated PSC-CMs were replated at day 9 for the experiments at day 10 or replated around day 20 for the experiments at day 30. Whole cell C²⁺ transients were measured as described above. Human left ventricular myocytes were isolated from donor hearts that were not suitable for transplantation as described previously (Chen et al., 2002 Circ Res, 91, 517-524.) and isolated myocytes were loaded with Fluo-3 AM for measurements. Myocytes were placed in a heated chamber on the stage of an inverted microscope (Nikon Diaphot). The chamber was superfused with Tyrode's solution. Myocyte was monitored with video-edge detection and intracellular Fluo-3 fluorescence was recorded with Clampex (Molecular Devices) as described (Piacentino et al., 2003 Circ Res, 92: 651-658). Data were analyzed offline with pClampfit.

### Transmission Electron Microscopy

Transmission electron microscopy (TEM) was performed with 1-month-old in vivo-matured mESC-CMs and ARVC iPSC-CMs. Sources of human control and ARVC heart sections were described (Basso et al., 2006 Eur Heart J, 27: 1847-1854). Heart tissue was fixed with freshly made EM grade 1% glutaraldehyde (Pella), 80 mM phosphate buffer (Sorenson's) and 3 mM magnesium chloride pH 7.2 at 4 C for 1 hour. To ensure complete fixation, tissues were then microwaved in a Pelco 3400 laboratory grade microwave oven. Tissues were placed in 4 ml uncapped glass vials containing 2 mls of fixative. The vials were then placed in a shallow ice bucket with the top of the fixative level equal to the top ice level. Two 600 ml beakers containing room temperature D-H2O were positioned on either side of the ice bucket to serve as heat traps. Samples were microwaved pulsed for 10 sec, paused for 20 sec, then pulsed again for 10 sec. Tissues were allowed to sit in fixative for 5 min, then microwaved again in the same manner Fixative temperatures never exceeded 27° C. Samples were rinsed in buffer containing 3% sucrose (3×15 min.), then microwaved twice again as before in secondary fixative. This osmication was performed in 1.5% potassium ferrocyanide reduced 1% osmium tetroxide in 100 mM phosphate buffer, containing 3 mM magnesium chloride. All subsequent steps were performed at 4° C. Tissues were then rinsed in 100 mM maleate buffer (3×5 min) containing 3% sucrose, then en-bloc stained with 1% filtered uranyl acetate in the same buffer for 1 hr. Samples were dehydrated at 4° C. up to 70% ethanol when they were brought to room temp and further dehydrated to 100% ethanol. Samples were embedded with Eponate 12 after a brief acetone transition, and finally cured in a 60° C. oven for two days. 80 nm ultra-thin sections were picked up on formvar coated 200 mesh nickel grids. Sections were floated on all subsequent steps. All solutions were filtered except for Abys, which were centrifuged at 13K for 5 min Grids were placed on 3% sodium metaperiodate (aq) for 20 min After a 15 min D-H₂O rinse, grids were placed on 50 mM NH4Cl in TBS for 10 min, followed by 20 min triple serum block (3% NGS, 3% BSA 1% fish gelatin) in TBST (blocking solution). GFP antibody (mouse) incubation was done at 1:200 with no primary antibody as negative controls. Incubations were carried out at 4° C. overnight. After 1 hr to equilibrate to room temperature, grids were placed on blocking solution for 10 min, followed by a 1 min rinse in TBS. 12 nm GAM (Gold conjugated secondary antibody, Jackson Immunoresearch) were diluted 1:40 in TBS and grids were incubated for 2 hrs at room temperature in a humidity chamber. After a 10 min TBS incubation followed by a quick D-H20 rinse, grids were hard fixed in 1% glutaraldehyde in 100 mM sodium cacodylate buffer for 5 min After a brief D-H₂O rinse, grids were stained with 2% uranyl acetate (aq.) for 20 min, rinsed again with D-H₂O; blot dried an allowed to sit in grid boxes overnight before viewing.

### Library Preparation and Sequencing

Single CMs (aMHC-GFP) were either FACS sorted (SH800, Sony Technologies) or manually picked under the microscope into 96-plates containing water (2.4 μH) with RNase-free DNase I (0.2 μl; NEB) and RNase inhibitor (0.25 μi; NEB). DNase I was inactivated by increasing the temperature (72° C. for 3 min), and samples were then stored on ice. Custom designed 2A oligo 1 μl primer (12 μM, Integrated DNA Technologies (Shin et al., 2015 Cell Stem Cell, 17: 360-372) was added and annealed to the polyadenylated RNA by undergoing a temperature increase (72° C. for 2 min) and being quenched on ice. A mixture of 1 μR SMARTscribe reverse transcriptase (Clontech Laboratories, Inc), 1 μL custom designed TS oligo (12 μM, Integrated DNA Technologies (Shin et al., 2015 Cell Stem Cell, 17: 360-372), 0.3μ MgCl₂ (200 mM, Sigma), 0.5μ RNase inhibitor (Neb), 1 μi dNTP (10 mM each, Thermo), 0.25 μe DTT (100 mM, Invitrogen) were incubated at 42° C. for 90 min, which was followed by enzyme inactivation at 70° C. for 10 min. A mixture of 29μ water, 5 μw Advantage2 taq polymerase buffer, 2 μp dNTP (10 mM each, Thermo), 2 μe custom-designed PCR primer (12 μM, Integrated DNA Technologies (Shin et al., 2015), and 2μ Advantage2 taq polymerase was directly added to the reverse transcription product and the amplification was performed for 19 cycles. The amplification product was purified using Ampure XP beads (Beckman-Coulter).

### Library

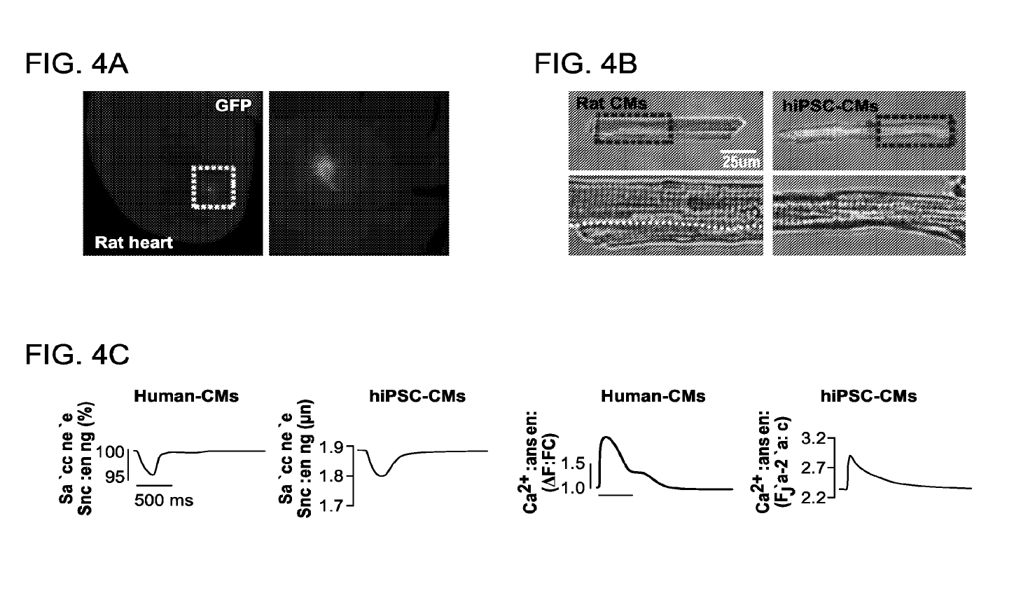

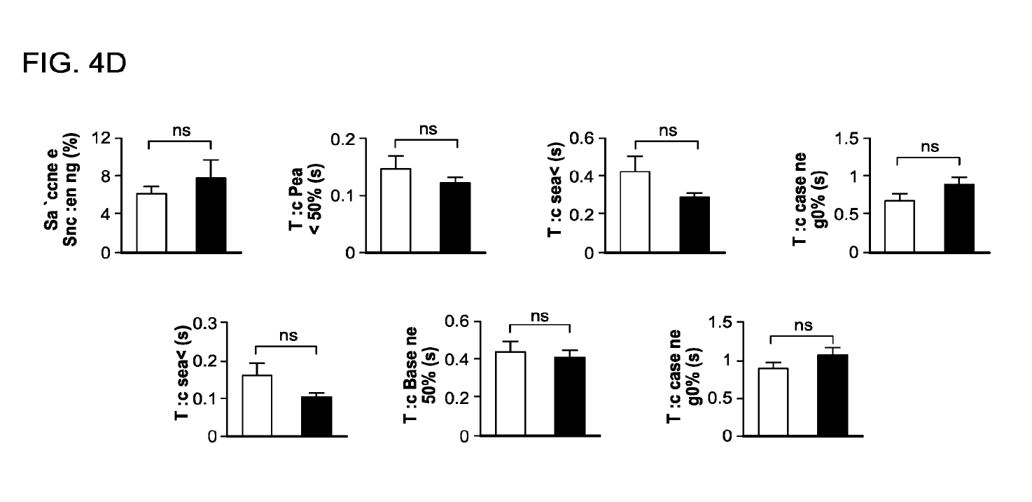

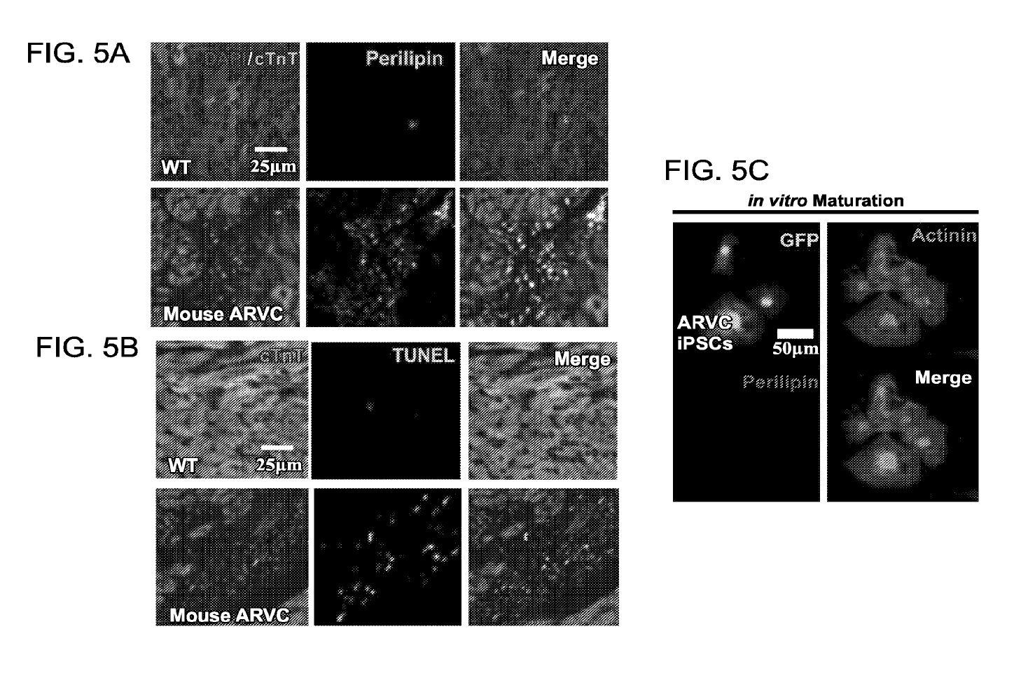

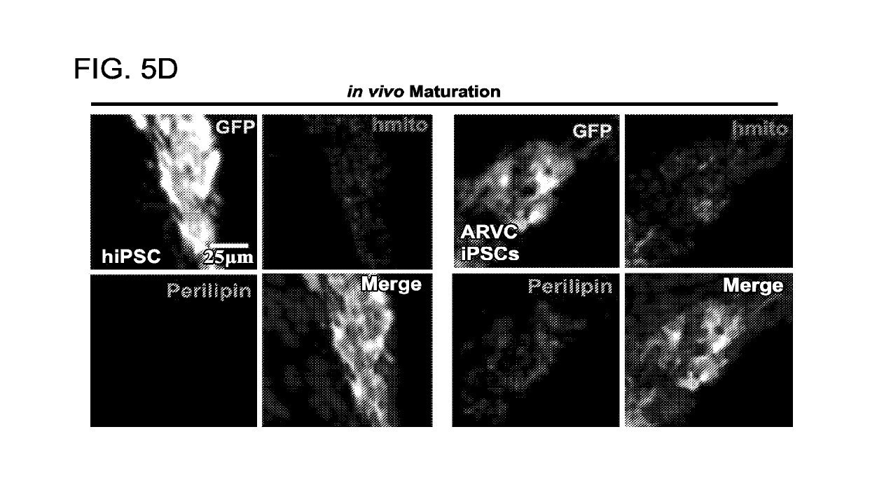

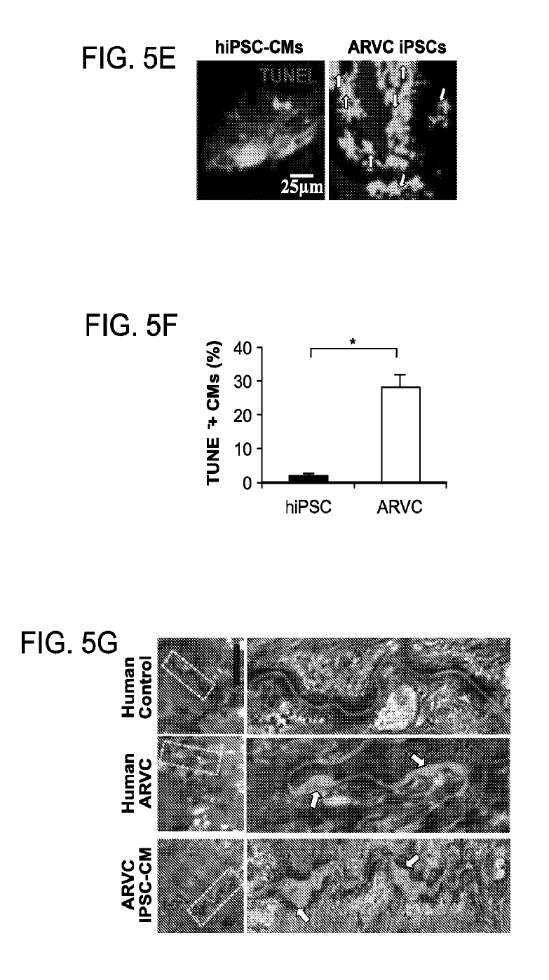

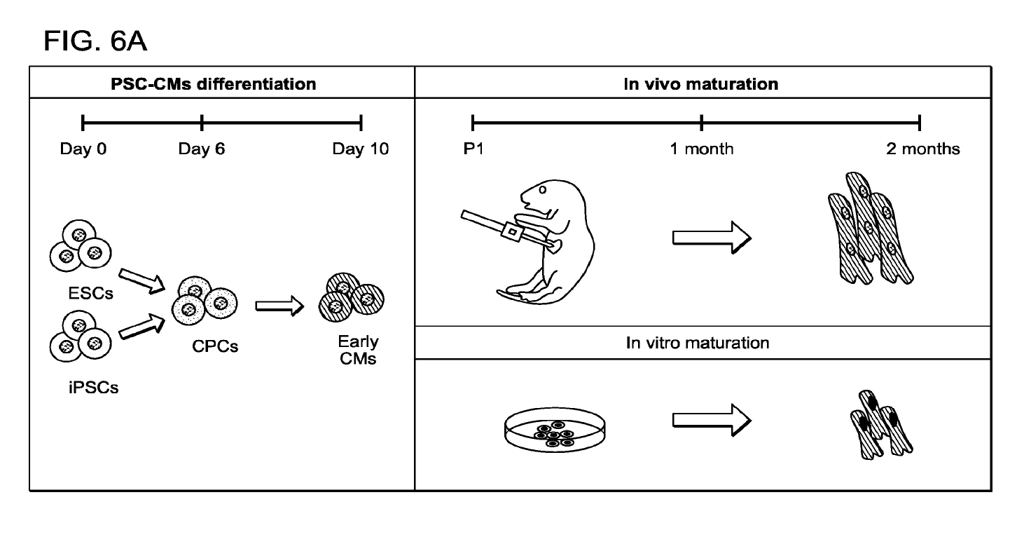

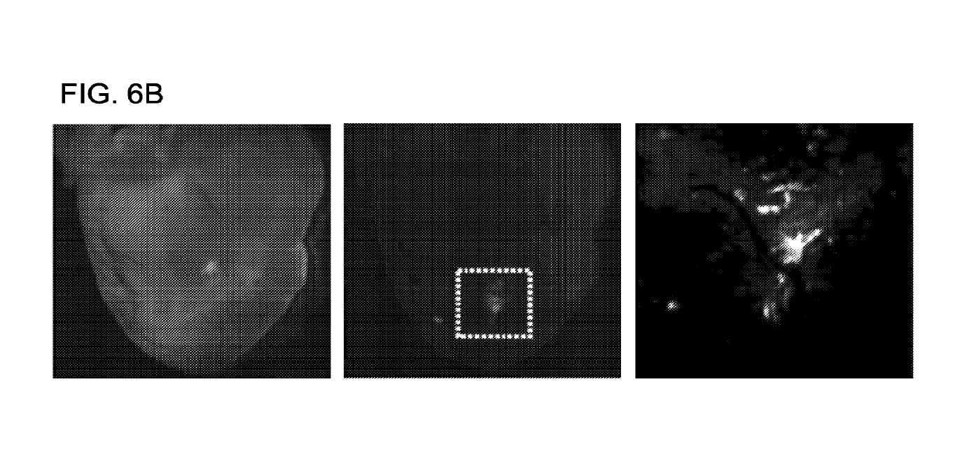

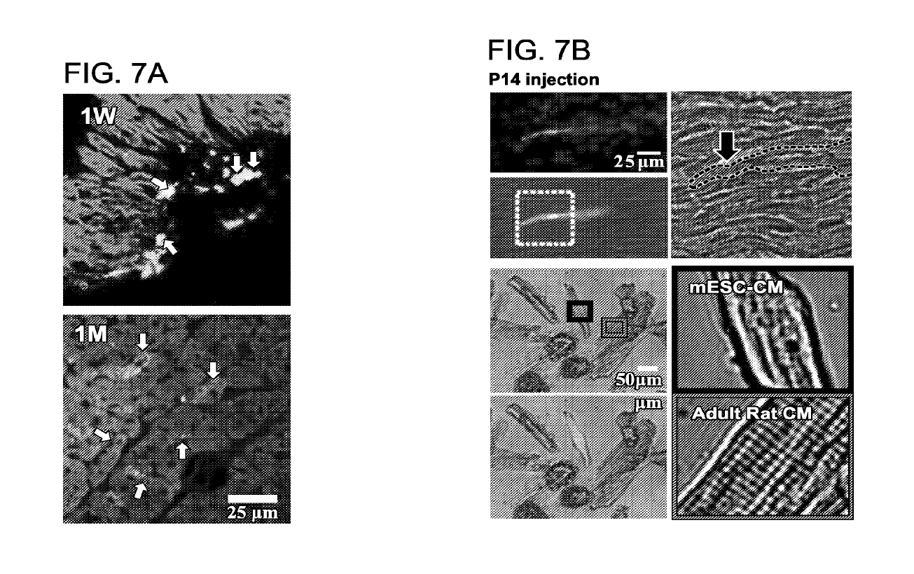

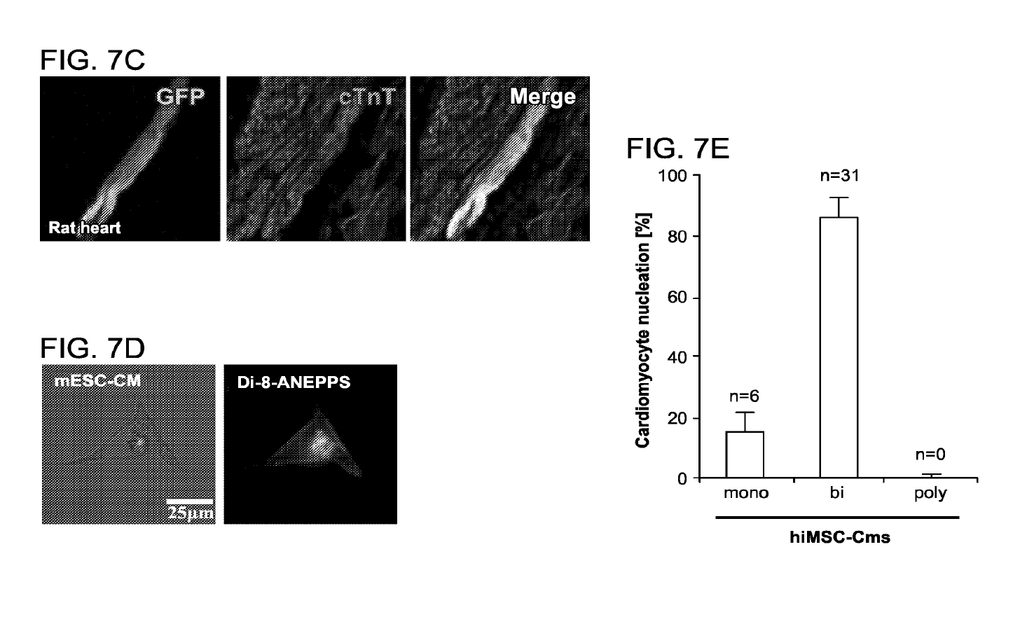

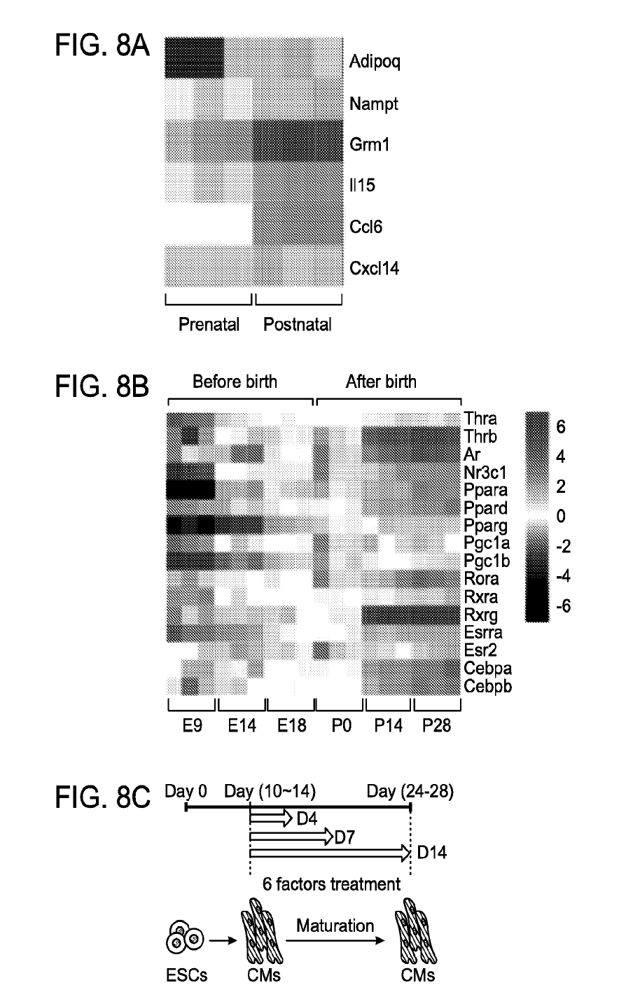

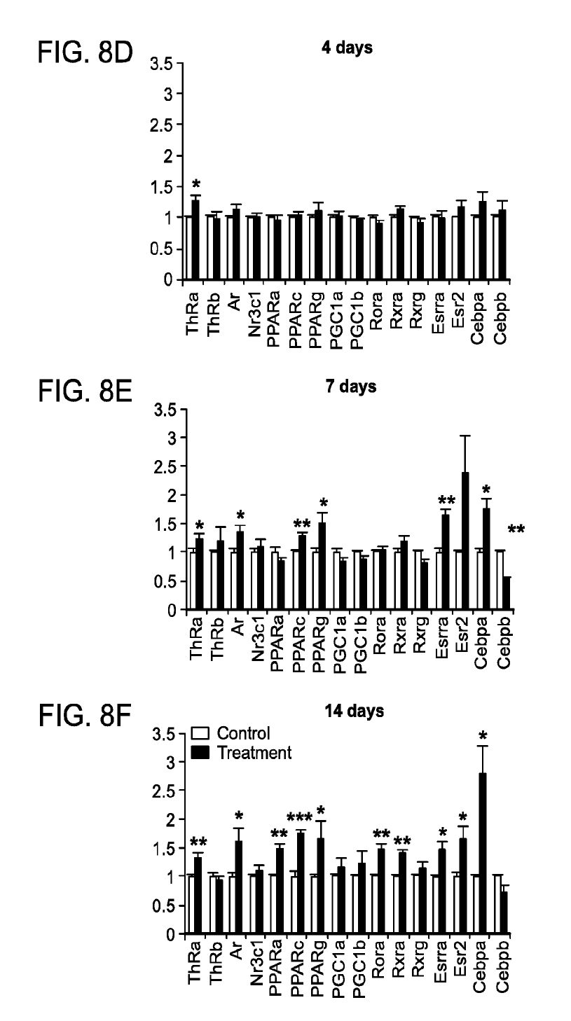

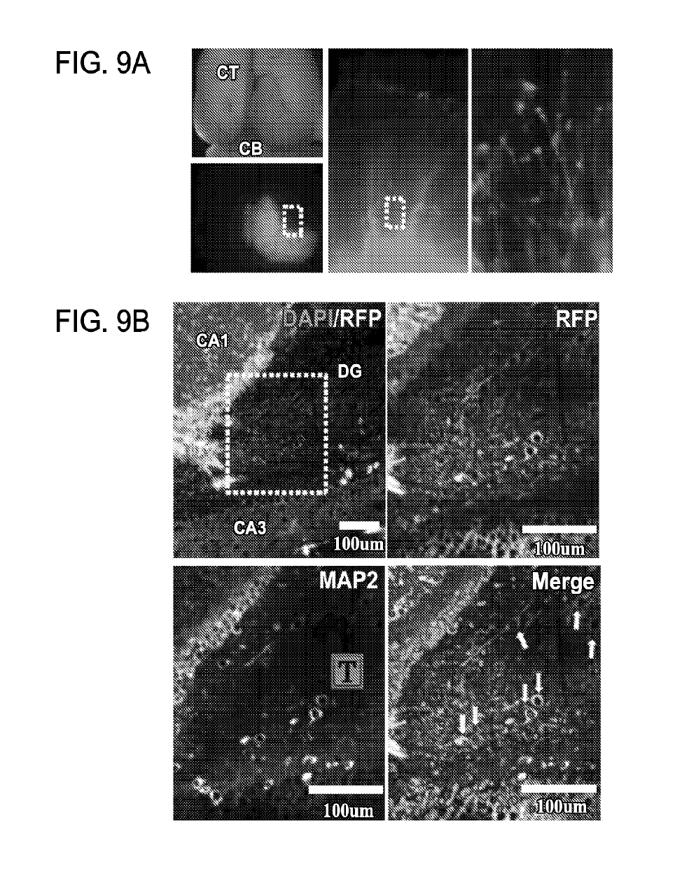

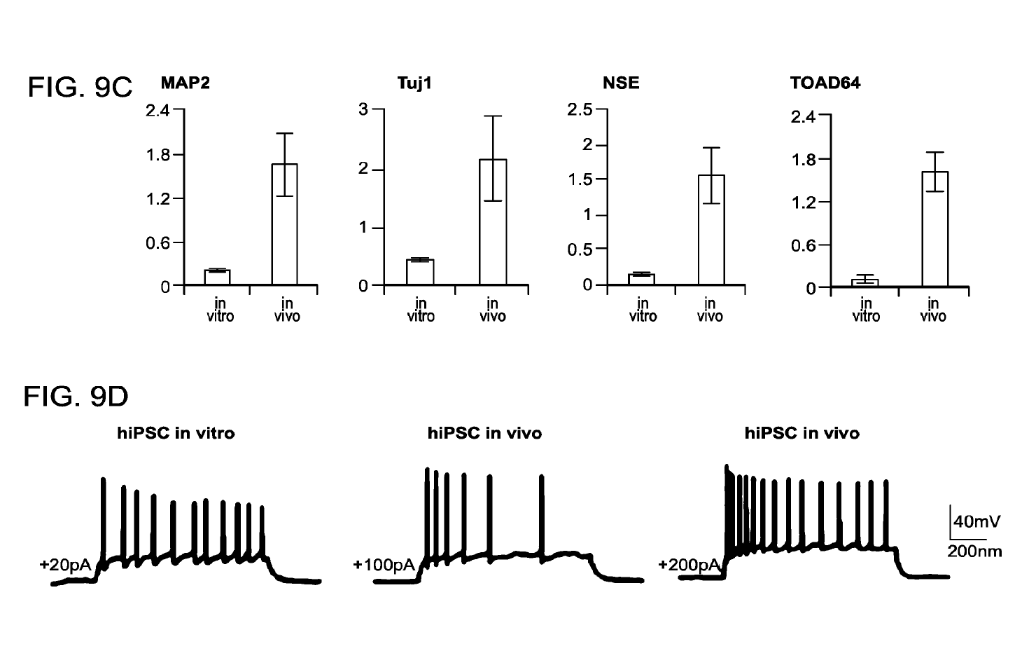

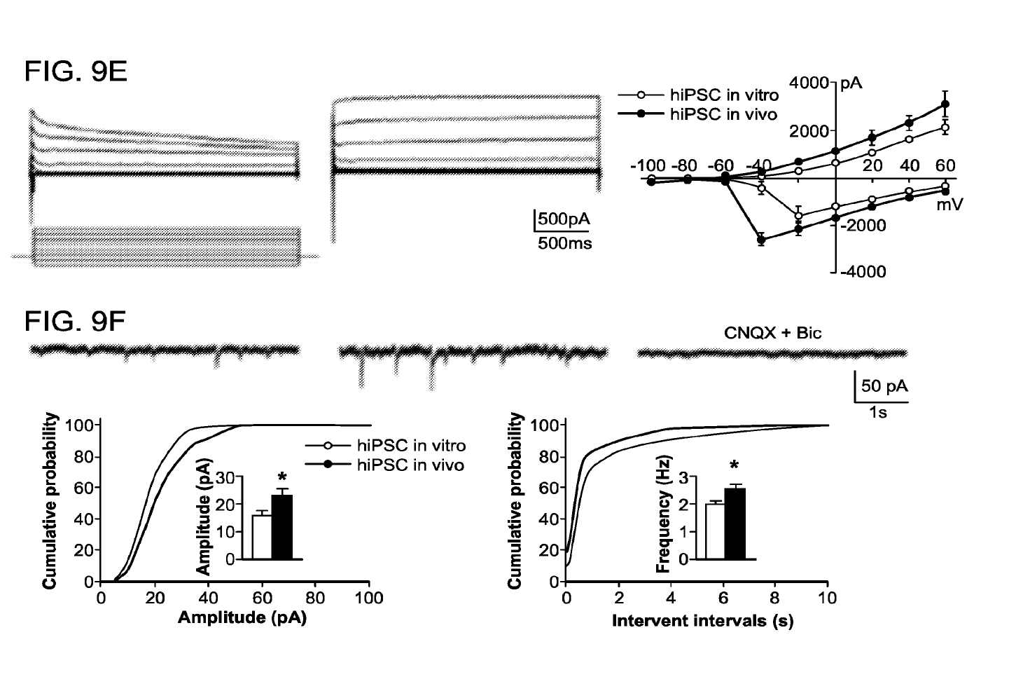

Libraries and transposome assembly where made using a previously published protocol (Picelli, et al., 2014 Nature protocols, 9: 171-181). Briefly, 100 pg of total cDNA was added to a 2× tagment DNA Buffer (TD) (2× TAPS buffer: 20 mM TAPS-NaOH, 10 mM MgCl2 (pH 8.5) at 25° C., 16% w/v PEG 8000) then spiked with 0.5 μl of 1:64 diluted Tn5 (Epicenter) and incubated for 8 min at 55° C. Tn5 was stripped off from the tagmented DNA, by adding 0.2% SDS for a final concentration of 0.05%. Libraries were enriched used KAPAHiFi which included 5× Kappa Fidelity Buffer, 10 mM dNTPs, HIFI Polymerase and lul of index primers was used directly in the enrichment PCR amplification of libraries for the Illumina sequencers for a 50 μl reaction. The PCR program was as follows: 5 min at 72° C., 1 min at 95° C., then 16 cycles at 30 sec at 95° C., 30 sec at 55° C., 30 sec at 72° and 5 min at 72°. Successful libraries were multiplexed and sequenced using NextSeq 500. For analysis, Trimmed reads (FIG. 4A) were mapped to the mouse reference genome (GRCm38/mm10) using Tophat (2.1.0) (Kim et al., 2013 Genome biology, 14: R36). Cells with >100,000 aligned reads were assembled in python package HTseq (Anders et al., 2015 Bioinformatics, 31: 166-169) then analyzed for differential expression in the DESeq2 package (Love et al., 2014 Genome biology, 15: 550) in R. Gene ontology analysis results were visualized using Revigo (Supek et al., 2011).

### Binucleation Analysis

CMs were isolated from rat hearts using the Langendorff technique. Cells were stained using antibodies against RFP (ChromoTeck), GFP and cTnT (Thermo Fisher) and DAPI. Cells were analyzed by FACS (SH800, Sony Biotechnologies) and by microscope (EVOSfl, AMG) to determine nucleation status.

### TUNEL Staining

In Situ Cell Death Detection Kit, TMR red (Roche Applied Science, catalog number 12156792910) was used for TUNEL staining.

### Statistical Analyses

For all analyses, sample size is described in the Brief Description of the Drawings. Most of in vitro studies were done with two to three sets of independent experiments. Two-group analysis used either Student's t-test or non-parametric Mann-Whitney test. Comparisons of multiple groups were performed using either One-way or Two-way ANOVA (if appropriate). If normality or equal variance tests failed, then a Kruskal Wallis test was used. Post-hoc multiple comparisons testing used either a Turkey's or Dunn's test. P<0.05 was considered significant.

### Example 2: In Vivo-Matured PSC-CMs are Morphologically Indistinguishable from Adult CMs

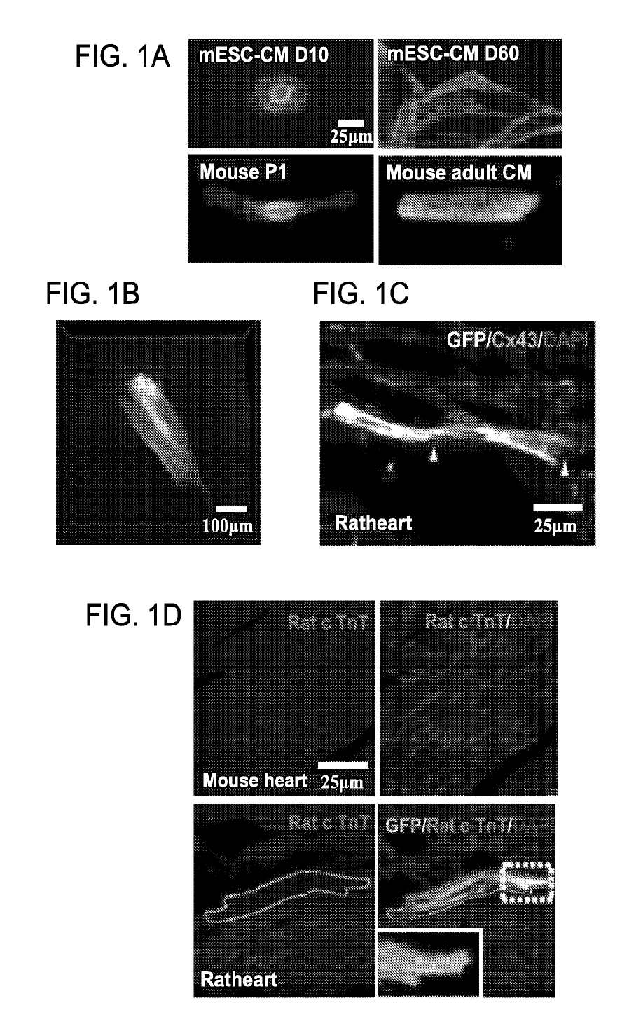

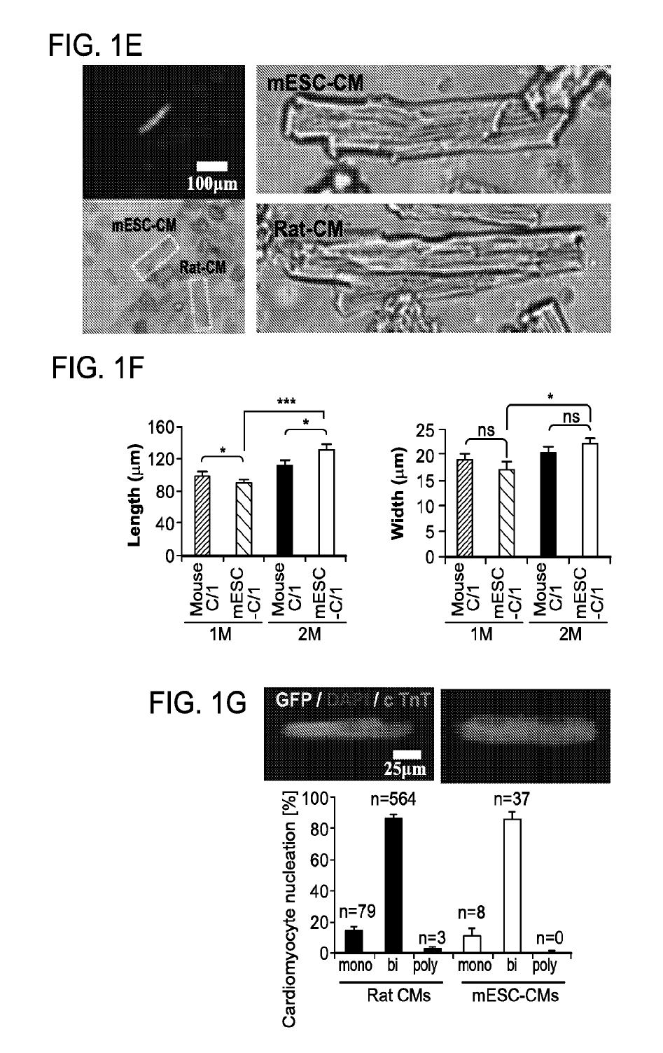

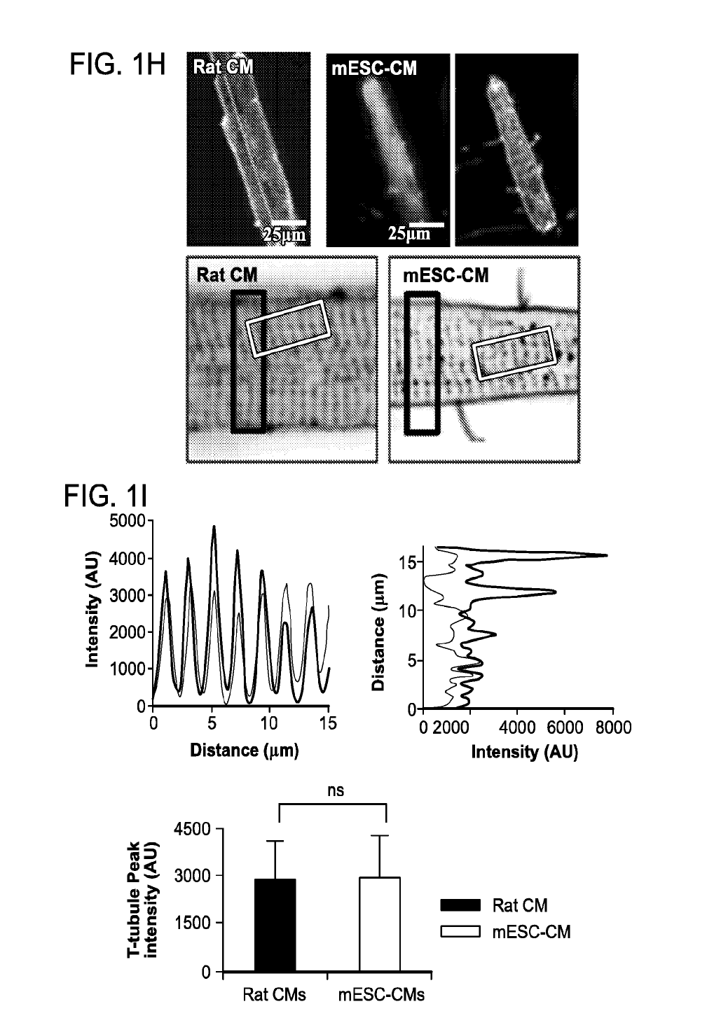

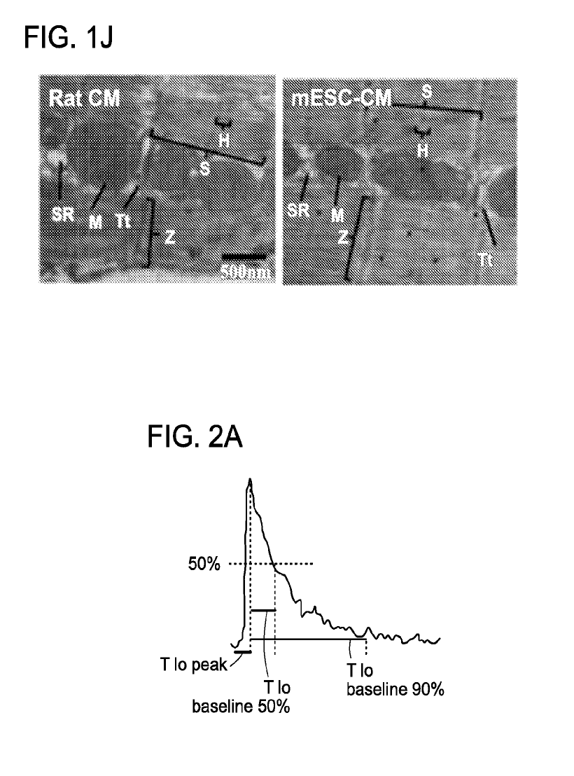

To examine the morphology of PSC-CMs in long-term culture, mouse embryonic stem cells (mESCs) were differentiated into CMs by sequential differentiation of ESCs into mesoderm, cardiac progenitor cells (CPCs), and CMs (Cheng et al., 2013 Development, 140: 2587-2596; Uosaki et al., 2012 Direct Contact with Endoderm-like Cells Efficiently Induces Cardiac Progenitors from Mouse and Human Pluripotent Stem Cells. PLoS One.). The resulting CMs were cultured in a condition shown to enhance CM maturation (Lundy et al., 2013 Stem Cells Dev, 22: 1991-2002). The mESC-derived CMs (mESC-CMs) increased in size over time, but remained mononucleated and irregular in shape with cytoskeletal disarray, which is similar to neonatal CMs, but distinct from adult CMs that are cylindrical with a well-organized cytoskeleton (FIG. 1A). This is consistent with a previous finding that in vitro-matured PSC-CMs are arrested at a neonatal stage at the molecular level (Uosaki et al., 2015 Cell reports, 13: 1705-1716).

Based on the neonatal arrest, it was reasoned that neonatal environment might possess the capability to mature PSC-CMs. Since neonatal hearts contain Isl1⁺ CPCs that was shown to become adult CMs without cell fusion (Laugwitz et al., 2005 Nature, 433: 647-653; Zaruba et al., 2010 Circulation, 121: 1992-2000), it was examined if PSC-derived Isl1⁺ CPCs (PSC-CPCs) could become mature CMs along with host CPCs/CMs in neonatal hearts. To do this, a mESC^{Isl1-Cre; Rosa-RFP; aMHC-GFP} line that expresses red fluorescent protein (RFP) constitutively in Isl1⁺ CPCs and green fluorescent protein (GFP) in CMs was generated, which allows tracing mESC-CPCs and monitoring their differentiation into CMs (Shenje, et al., 2014 Elife 3, e02164). RFP⁺ CPCs at day 6 were purified by fluorescence-activated cell sorting (FACS) and their development was monitored following their intraventricular delivery (˜200K cells/injection) at postnatal days (P) 1-3 (FIG. 6A). To avoid immune rejection, NIH nude rats (Liang et al., 1997 Lab Anim Sci, 47: 549-553) were used. On average, ˜2,000 RFP⁺ cells were obtained per site of injection, which was sufficient for subsequent in vivo and in vitro analyses. At 1 week of incubation, the CPCs expressed GFP, suggesting their differentiation into CMs, but they were spherical with a single nucleus (FIG. 7A). However, they became similar to adult CMs in morphology after 1 month of incubation (FIG. 7A). A 3D reconstruction of the heart, generated with a tissue-clearing method (Susaki et al., 2014 Cell, 157: 726-739), revealed that the incubated cells form adult CM-like patches (FIG. 1B). Connexin43, a gap junction protein, was expressed in the mESC-CMs, indicating coupling with neighboring CMs (FIG. 1C). The mESC-CMs were not detected by rat cardiac troponin T (cTnT) antibody that does not cross-react with mouse CMs (FIG. 1D), excluding the possibility of cell fusion. These data suggest that early postnatal hearts are capable of maturing mESC-CMs. However, the capability appears to be lost when mESC-CMs are incubated from P14 (FIG. 7B), implying the presence of a window similar to that observed for neonatal heart regeneration following tissue excision (Porrello et al., 2011 Science, 331: 1078-1080).

To analyze the morphology of in vivo-incubated PSC-CMs in detail, they were isolated via Langendorff perfusion and compared with endogenous CMs. The mESC-CMs, identified by GFP expression, had well-organized sarcomere structure as control adult CMs (FIG. 1E). The length and width of 1-month-incubated mESC-CMs were similar to those of 1-month-old mouse CMs. 2-month-old mESC-CMs were slightly bigger than 1-month-old ones, but similar to 2-month-old mouse CMs in size (FIG. 1F). Similar to adult CMs, the mESC-CMs exhibited a high level of binucleation, an indicator of CM maturation (FIG. 1G).

Formation of transverse (T)-tubules, invaginations of the plasma membrane essential for excitation-contraction coupling in adult CMs, is considered a structural hallmark of CM maturation (Yang et al., 2014 Circ Res, 114: 511-523). In rats, T-tubules appear sparsely around 2 weeks after birth and fully develop by the first month (Ziman et al., 2010 J Mol Cell Cardiol, 48: 379-386). To determine if the in vivo-incubation allows T-tubule formation, mESC-CMs were isolated after 1 month of incubation in vivo and live-cell imaging with spinning-disk confocal microscopy was conducted. T-tubules were visualized by di-8-Annepps that labels plasma membranes (Kirk et al., 2003 The Journal of physiology, 547: 441-451). The mESC-CMs displayed a clear T-tubule pattern, which was absent in mESC-CMs matured in vitro (FIG. 1H, FIG. 7D). A detailed analysis showed evenly spaced T-tubules with peak intensities that are similar to host rat CMs (FIG. 1I), suggesting the presence of fully developed T-tubule networks. The presence of T-tubules was further confirmed by transmission electron micrographs, which revealed additional adult-like CM ultrastructures such as well-developed mitochondria and sarcomeres (FIG. 1J). The adult-like CMs were also generated by directly injecting early mESC-CMs.

### Example 3: In Vivo-Matured PSC-CMs Exhibit Calcium Transients and Contractility of Adult CMs

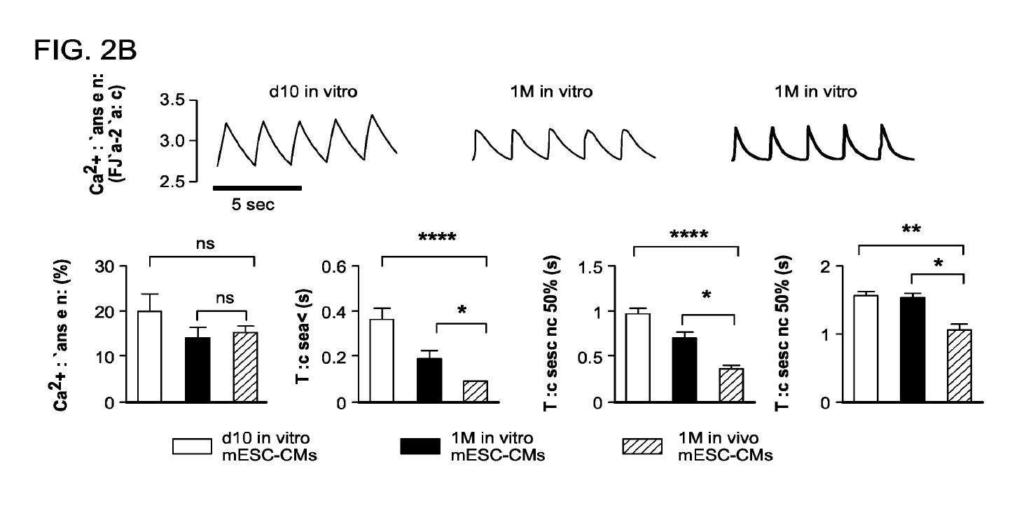

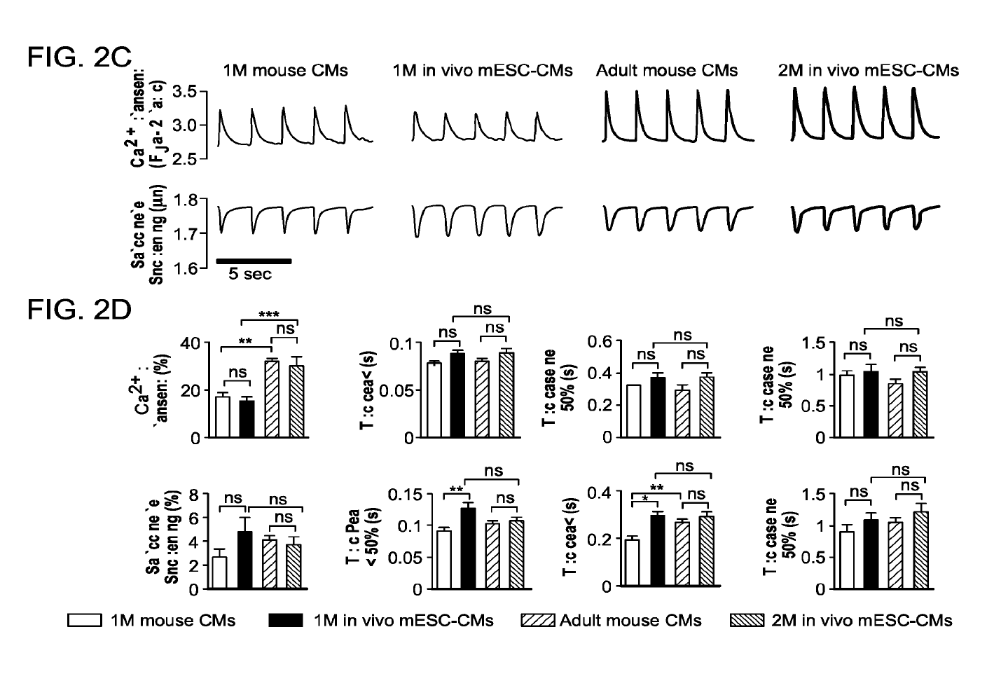

To determine if the morphological maturation of mESC-CMs is accompanied by functional maturation, we measured C²⁺ transients of mESC-CMs matured in the heart and culture using the ratiometric dye Fura-2 AM. The in vivo-incubated mESC-CMs showed peak amplitude of Ca²⁺ transients similar to cultured mESC-CMs. However, their time to peak amplitude and return to baseline was significantly shorter than cultured mESC-CMs (FIG. 2A, FIG. 2B and Table 1). This indicates that in vivo-incubated mESC-CMs develop more mature Ca²⁺ transients than mESC-CMs cultured for 10 or 30 days. The amplitude of Ca²⁺ transients at 1- or 2-month old in vivo-maturated CMs was increased compared to control CMs at corresponding stage (1- or 2-month). However, compared to endogenous CMs, in vivo-matured mESC-CMs showed similar levels of time to peak and 50% and 90% relaxation. (FIG. 2C, FIG. 2D, and Table 1).

TABLE 1
Summary of Sarcomere Shortening and Ca2+ Transients for Mouse CMs and mESC-CMs

| | Sarcomere shortening | | | | Ca²⁺ transients | | | |
|---|---|---|---|---|---|---|---|---|
| | Peak | T to peak 50% | T to Peak | T to bl 90% | Peak | T to Peak | T to bl 50% | T to bl 90% |
| | (%) | (sec) | (sec) | (sec) | (%) | (sec) | (sec) | (sec) |
| d 10 | NA | NA | NA | NA | 19.961 ± 3.525 | 0.364 ± 0.041 | 0.966 ± 0.066 | 1.535 ± 0.074 |
| mESC-CMs | | | | | | | | |
| d 30 | NA | NA | NA | NA | 13.773 ± 2.556 | 0.183 ± 0.037 | 0.699 ± 0.069 | 1.513 ± 0.060 |
| mESC-CMs | | | | | | | | |
| 1 M mouse | 2.706 ± 0.631 | 0.092 ± 0.003 | 0.199 ± 0.013 | 0.914 ± 0.086 | 16.826 ± 2.001 | 0.078 ± 0.001 | 0.314 ± 0.007 | 0.989 ± 0.042 |
| CMs | | | | | | | | |
| 1 M in vivo | 4.687 ± 1.344 | 0.127 ± 0.009 | 0.296 ± 0.024 | 1.076 ± 0.126 | 15.064 ± 1.800 | 0.088 ± 0.003 | 0.370 ± 0.031 | 1.048 ± 0.096 |
| mESC-CMs | | | | | | | | |
| Adult Mouse | 4.058 ± 0.419 | 0.103 ± 0.003 | 0.207 ± 0.010 | 1.042 ± 0.074 | 31.786 ± 1.82 | 0.084 ± 0.002 | 0.299 ± 0.022 | 0.852 ± 0.072 |
| CMs | | | | | | | | |
| 2 M in vivo | 3.744 ± 0.608 | 0.108 ± 0.005 | 0.295 ± 0.020 | 1.228 ± 0.101 | 30.233 ± 3.611 | 0.089 ± 0.004 | 0.377 ± 0.022 | 1.032 ± 0.069 |
| mESC-CMs | | | | | | | | |

The contractile properties of the in vivo-matured mESC-CMs were further examined by measuring sarcomere shortening with optical video microscopy. This assay was not applicable for cultured PSC-CMs due to their disorganized sarcomere structure. The mESC-CMs and endogenous CMs showed similar characteristics in the amplitude of sarcomere shortening to endogenous CMs at corresponding stages. The mESC-CMs showed slower velocities in both the time to peak and the time to peak 50% at 1 month, but the velocity became insignificant at 2 months (FIG. 2C, FIG. 2D and Table 1). However, no significant difference was observed in relaxation among the stages.

### Example 4: In Vivo-Matured PSC-CMs Are Similar to Adult CMs in Gene Expression

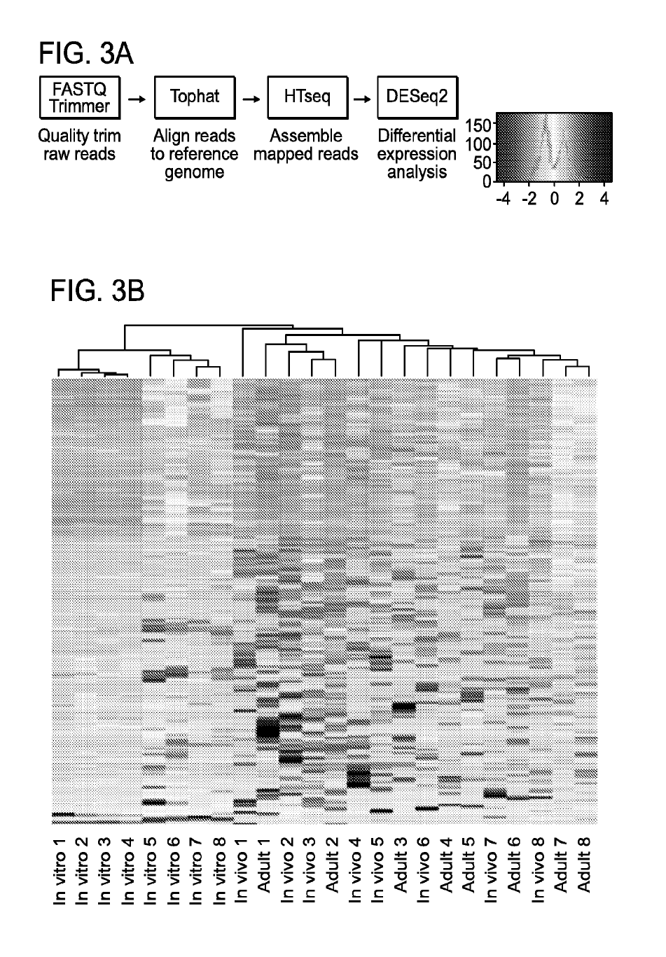

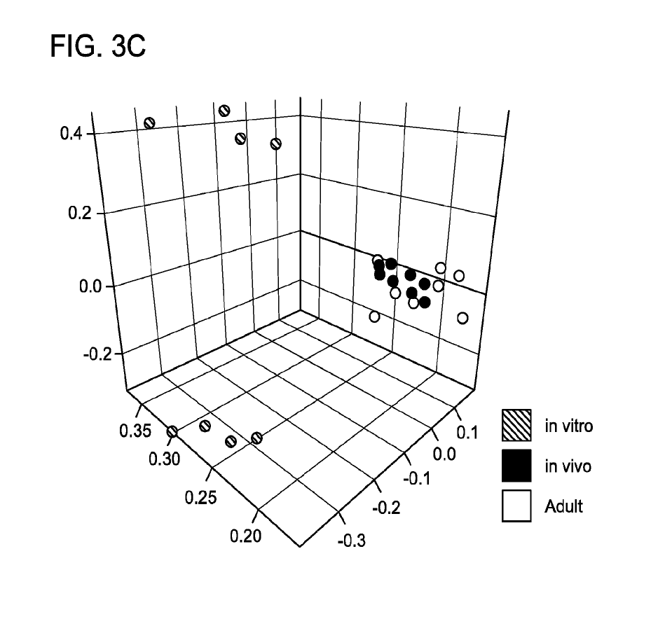

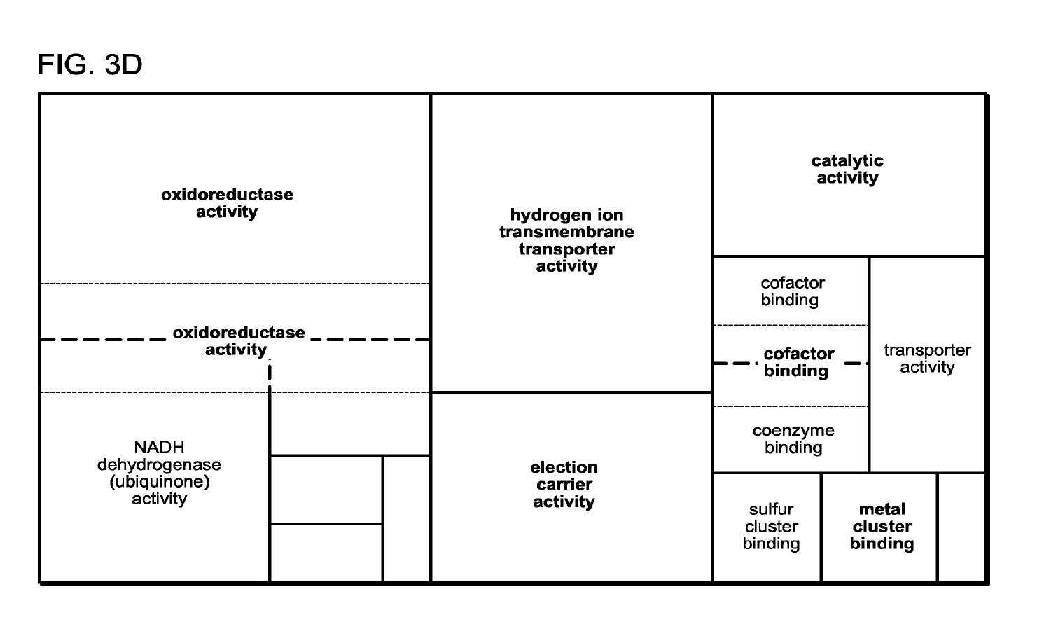

The transcriptomes of mESC-CMs matured were characterized in vivo and in vitro. To do this, the CMs were isolated from hearts or culture after 1 month of incubation and subjected to single-cell RNA-sequencing analysis (FIG. 3A). Mouse adult CMs were used as a control group. 304 differentially expressed genes (>twofold change) were identified based on a P-value less than 0.05 with an adjusted P-value less than 0.1. Hierarchical clustering analysis revealed that in vivo-matured CMs clustered closer to adult CMs than in vitro-matured CMs (FIG. 3B). Consistently, adult and in vivo-matured CMs were grouped closer than to in vitro-matured CMs in principal component analysis (FIG. 3C). These suggest similarity in gene expression between adult and in vivo-matured CMs. Notably, in vitro-matured CMs appear to have two distinct subpopulations (FIG. 3C). This may be attributed to variability in their differentiation and maturation in vitro. Gene ontology (GO) analysis indicated that groups of the differentially expressed genes were related to mitochondrial function and metabolic pathways (FIG. 4C). This may reflect the increase in mitochondrial biogenesis and fatty acid oxidation during maturation. In addition, GO analysis noted an increase in contractile proteins previously shown to increase during cardiac maturation.

### Example 5: Postnatal Extracellular Factors Can Promote PSC-CM Maturation In Vitro

The fact that PSC-CMs do not mature beyond neonatal stages in culture (Uosaki et al., 2015 Cell reports, 13: 1705-1716), but can be further matured in neonatal hearts implies environmental differences between postnatal and prenatal hearts. To gain insights into the environmental potential of postnatal hearts, genes expressed differentially during CM maturation were analyzed and 89 genes were identified using the affymetrix array datasets (Uosaki et al., 2015 Cell reports, 13: 1705-1716). Of those, 25 were highly downregulated and 16 were highly upregulated in the postnatal heart. Genes encoding secreted or membrane-bound proteins were focused on and it was identified that 6 of those [CXCL14 (C-X-C Motif Chemokine Ligand 14), IL-15 (Interleukin 15), CCL16 (C-C Chemokine ligand 16), Adipoq (Adiponectin), Grm1 (Glutamate Metabotropic Receptor 1), Nampt (Nicotinamide phosphoribosyltransferase)] were increased in postnatal hearts, determined by qPCR (FIG. 8A). Subsequently, mESC-CMs were treated with activators (P7C3-activator of Nampt and DHPG -Glutamate Receptor agonist) and recombinant proteins (CXCL14, IL-15, CCL16 and ADIPOQ) of those factors for different time intervals and their maturation status was assessed (FIG. 8C). To generate a genetic readout for the assessment, Ingenuity pathway analysis was utilized and a group of nuclear receptors (NRs) strongly associated with the postnatal heart maturation (FIG. 8B) was identified. Their expression levels were verified by qPCR. Upon activating the factors, the majority of NRs were significantly upregulated after 7 days of culture (FIG. 8D and FIG. 8E). More NRs were increased over time and after 14 days of culture (FIG. 8F). These suggest that the extracellular proteins may be an environmental component of postnatal hearts utilized for CM maturation.

TABLE 3
Primer list for quantitative PCR

| | | SEQ ID | | SEQ ID |
|---|---|---|---|---|
| Gene | Forward Primer | NO: | Reverse Primer | NO: |
| Cxcl14 | gacagacggcaggagcac | 1 | tttcaagcacgcctctctc | 24 |
| Ccl6 | tctttatccttgtggctgtcc | 2 | tggagggttatagcgacgat | 25 |
| Il15 | cagaggccaactggatagatg | 3 | actgtcagtgtataaagtggtgtcaat | 26 |
| Adipoq | ggagagaaaggagatgcaggt | 4 | ctttcctgccaggggttc | 27 |
| Nampt | ggcagaagccgagttcaa | 5 | tgggtgggtattgtttatagtgag | 28 |
| Grm1 | tttacctgcagagcctgtga | 6 | tccactcgaggtaacggatag | 29 |
| Rora | cagagcaatgccacctactcct | 7 | ctgcttcttggacatccgacca | 30 |
| Rxra | gtgaaagatgggattctcctggc | 8 | gtcacgcatcttagacaccagc | 31 |
| Esrra | actacggtgtggcatcctgtga | 9 | ggtgatctcacactcattggagg | 32 |
| Esr2 | ggtcctgtgaaggatgtaaggc | 10 | taacacttgcgaagtcggcagg | 33 |
| Thra | cctggacaaagacgagcagtgt | 11 | ctggattgtgcggcgaaagaag | 34 |
| Thrb | accactatcgctgcatcacctg | 12 | actggttgcgggtgactttgtc | 35 |
| Nr3c1 | agctccccctggtagagac | 13 | ggtgaagacgcagaaaccttg | 36 |
| Ppara | accactacggagttcacgcatg | 14 | gaatcttgcagctccgatcacac | 37 |
| Ppard | ggaccagaacacacgcttcctt | 15 | ccgacattccatgttgaggctg | 38 |
| Pparg | gtactgtcggtttcagaagtgcc | 16 | atctccgccaacagcttctcct | 39 |
| Pgc1a | gaatcaagccactacagacaccg | 17 | catccctcttgagcattcgtg | 40 |
| Pgc1b | cccagcgtctgacgtggacgagc | 18 | ccttcagagcgtcagagcttgctg | 41 |
| Rxrg | ttgtctcatcgacaagcgccag | 19 | ctggcacattctgcctcactct | 42 |
| Ar | ctgggaagggtctacccac | 20 | ggtgctatgttagcggcctc | 43 |
| Cebpa | aatggcagtgtgcacgtcta | 21 | ccccagccgttagtgaagag | 44 |
| Cebpb | caacctggagacgcagcacaag | 22 | gcttgaacaagttccgcagggt | 45 |
| Gapdh | tgtgtccgtcgtggatctga | 23 | ttgctgttgaagtcgcaggag | 46 |

### Example 6: Human PSC-CMs Are Matured to Adult-like CMs in Rodent Neonatal Hearts

Human CMs are analogous to rodent CMs in size and structure, but their maturation transpires over a decade. Since CM development is a conserved process in mammals, it was hypothesized that human iPSC-CMs (hiPSC-CMs) can be matured in rat postnatal hearts. To test this, hiPSCs (Takahashi K and Yamanaka S, 2006 Cell, 126: 663-676) were labeled with GFP, differentiated into CMs, and incubated in neonatal rat hearts as described earlier (FIG. 4A). Similar to mESC-CMs, hiPSC-CMs exhibited adult CM-like features after 1 month of incubation. They were rod-shaped with highly organized sarcomeres (FIG. 4B) and functionally similar to human adult CMs (FIG. 4C, FIG. 4D and Table 2). These data demonstrate that rodent neonates are capable of generating adult-like CMs from human PSCs. The hiPSC-CMs showed ˜80% binucleation (FIG. S2E), which is higher than that of human CMs (25-57%) reported.

TABLE 2
Summary of Sarcomere Shortening and Ca²⁺ Transients for Human CMs and hiPSC-CMs

| | Sarcomere shortening | | | | Ca²⁺ transients | | |
|---|---|---|---|---|---|---|---|
| | Peak | T to peak 50% | T to Peak | T to bl 90% | T to Peak | T to bl 50% | T to bl 90% |
| | (%) | (sec) | (sec) | (sec) | (sec) | (sec) | (sec) |
| Adult Human | 6.097 ± 0.904 | 0.146 ± 0.022 | 0.422 ± 0.080 | 0.681 ± 0.088 | 0.160 ± 0.034 | 0.447 ± 0.053 | 0.896 ± 0.067 |
| CMs | | | | | | | |
| Human iPSC-CMs | 7.659 ± 2.097 | 0.120 ± 0.010 | 0.290 ± 0.019 | 0.877 ± 0.109 | 0.104 ± 0.008 | 0.417 ± 0.032 | 1.059 ± 0.090 |
| In vivo maturation | | | | | | | |

### Example 7: In Vivo-Maturation System Allows Modeling Human Arrhythmogenic Right Ventricular Cardiomyopathy (ARVC)

ARVC is an inherited form of cardiomyopathy that manifests in adolescence/adulthood in humans, and is characterized by fibro-fatty replacement, apoptosis and intercalated disc abnormalities (Basso et al., 2006 Eur Heart J, 27: 1847-1854; Calkins H and Marcus F, 2008 Curr Cardiol Rep, 10: 367-375). The adult-specific phenotype was confirmed in our mouse ARVD model (FIG. 5A and FIG. 5B). With the importance of the disease, ARVC hiPSCs were generated from human patients (Kim et al., 2013 Nature, 494: 105-110). However, the resulting ARVC hiPSC-CMs remained fetal-like in morphology and function, and required a lipogenic medium to partially mimic ARVC properties (Kim et al., 2013 Nature, 494: 105-110). It was also identified that ARVC hiPSC-CMs matured in vitro do not show the disease phenotype (FIG. 5C). To test if the neonatal system can be used to model human ARVC, GFP-labeled ARVC hiPSC-CMs were introduced into neonatal rat hearts and analyzed them after 1 month of incubation. The incubation led to an accumulation of lipids/adipocytes, accompanied with markedly increased apoptosis (FIG. 5D-FIG. 5F), recapitulating the disease phenotype. Moreover, electron microscopy showed abnormal intercalated discs (intercellular gap widening) observed in ARVC human patients (FIG. 4E).

### Example 8: Maturation of hiPSC-Derived Progenitors in the Brain

Next, it was determined if other neonatal organs, such as the brain, can mature hiPSC-derived progenitors. To do this, RFP-labeled hiPSCs were differentiated to neuronal progenitor cells (NPCs), introduced into P1 rat brains (200K cells/injection), and analyzed after 8 weeks. The hiPSC-NPCs were mostly retained in the brain (>90%) and formed clump/axon-like neuron structures (FIG. 9A). Sectional analysis revealed the contribution of RFP neurons in the cornu ammonis as well as the dentate gyms, a major area undergoing neurogenesis in the adult brain (FIG. 9B). Consistent with the morphological maturation, the RFP neurons expressed the mature neuron genes MAP2, Tuj1, NSE, and TOAD64 that were barely detected in hiPSC-neurons matured in vitro (FIG. 9B and FIG. 9C). The functional activity of hiPSC-neurons was further investigated by patch-clamp recordings after 8 weeks of maturation in vivo and in vitro. Cells in both cases were capable of firing repetitive action potentials in response to depolarizing current injection, but increasing action potential firing rate was observed with increasing current in hiPSC-neurons matured in vivo (FIG. 9D). They also showed voltage-dependent inward and outward currents upon application of depolarizing voltage steps (FIG. 9E). The currents were selectively sensitive to TTX or TEA, indicating the expression of functional sodium and potassium channels in cells. Robust spontaneous synaptic events were recorded in hiPSC-neurons matured in brains, with both amplitude and frequency significantly higher than in vitro-matured hiPSC-neurons (FIG. 9F). These data showed that in vivo-incubated hiPSC-neurons were able to display stereotypical neuronal behavior, including firing properties and extensive synaptic activity, suggesting their ability to integrate into the local neuronal circuits in vivo.

The following materials and methods were utilized in this example.

hiPSCs Culture and Neural Differentiation

hiPSC lines were maintained on inactivated mouse embryonic fibroblasts (MEFs). To reliably in vivo visualize and trace transplanted cells, stable hiPSC dsRED-SC1014, dsRED-SNCA cell lines were established by nucleofection with piggybac-dsRED transposon and piggybac transposase. All cell lines are maintained according standard protocol. Briefly, human pluripotent stem cells (hPSCs) were maintained in human ES cell medium containing DMEM/F12 (Invitrogen), 20% knockout serum replacement (KSR, Invitrogen), 4 ng/ml FGF2 (PeproTech), 1 mM Glutamax (Invitrogen), 100 μm non-essential amino acids (Invitrogen), 100 μM 2-mercaptoethanol (Invitrogen). Medium was changed daily. Cells were passaged using collagenase (1 mg/ml in DMEM/F12) at a ratio 1:6 to 1:12. All experiments using hESCs/iPSCs cells were conducted in accordance with the policy of the JHU SOM that research involving human pluripotent stem cells (hPSCs) being conducted by JHU faculty, staff or students or involving the use of JHU facilities or resources shall be subject to oversight by the JHU Institutional Stem Cell Research Oversight (ISCRO) Committee.

Neural differentiation of hiPSCs was based on rosette neural aggregates method (termed RONA, Xu et al., under revision). Briefly, to initiate differentiation, hPSCs colonies were allowed to incubate with Collagenase (1 mg/ml in DMEM/F12) in the incubator for about 5-10 min. The colony borders begin to peel away from the plate. The collagenase is gently washed off the plate with growth medium. While the colony center remains attached, the colonies were selectively detached with the MEFs undisturbed. Detached hPSC colonies were then growing as suspension in human ES cell medium without FGF2 for 2 days in low attachment 6-well plates (Corning). From day 2 to day 6, Noggin (50 ng/ml, R&D system) or Dorsomorphin Tocris) and SB431542 (10 μm, Tocris) were supplied in human ES cell medium (without FGF2, defined as KoSR medium). On day 7, free-floating EB were transfered to Matrigel or Laminin precoated culture plates to allow the complete attachment of EB aggregates with the supplement of N2-induction medium (NIM) containing DMEM/F12 (Invitrogen), 1% N2 supplement (Invitrogen), 100 μm MEM nonessential amino acids solution (Invitrogen), 1 mM Glutamax (Invitrogen), Heparin (2 μg/ml, Sigma). The culture was continually fed with N2-medium every other day from day 7-12. From day 12, N2-induction medium were changed every day. Attached aggregates will breakdown to form a monolayer colony on day 8-9 with typical neural specific rosette formation. With the extension of neural induction, highly compact 3-dimensional column-like neural aggregates (termed rosette neural aggregates, RONA) formed in the center of attached colonies. RONAs were manually microisolated taking special care to minimize the contaminating peripheral monolayer of flat cells, and cells underneath RONAs. RONA clusters were collected and maintained as neurospheres in Neurobasal medium (Invitrogen) containing B27 minus VitA (Invitrogen), 1 mM Glutamax (Invitrogen) for 1 day, the next day, neurospheres were dissociated into single cells and plated on Laminin/PDL coated plates for further experiments. For neuronal differentiation, either RA (2 μM), SHH (50 ng/ml), Purmorphamine (2 μM), or the combination of RA, SHH, Purmorphamine were supplemented in neural differentiation medium containing Neurobasal/B27 (NB/B27; Invitrogen), BDNF (brain-derived neurotrophic factor, 20 ng/ml; PeproTech), GDNF (glial cell line-derived neurotrophic factor, 20 ng/ml; PeproTech), ascorbic acid (0.2 mM, Sigma), dibutyryl cAMP (0.5 mM; Sigma) at indicated time after neurospheres were dissociated into single cells. For long-term neuronal culture, neural differentiation medium containing rat astrocyte-conditioned Neurobasal medium/B27, BDNF, GDNF, ascorbic acid, dibutyryl cAMP was used for maintenance.

Cell delivery

RNU Rats (Charles River Laboratories) were used as host animals. Postnatal rats were anesthetized by cooling on an ice bed. Cells were injected directly to the brain. Before injection, cells were mixed with IMDM and matrigel at 60:1 ratio27 and injected with Eppendorf FemtoJet® Microinjector (4 μl/injection).

### Electrophysiological Recording of Brain Slice

Transverse brain slices of 350 μm thickness were prepared at 8 weeks after differentiated hiPSC cells injection using a vibratome (Leica VT1200S). Slices were incubated in artificial cerebrospinal fluid (ACSF) containing (in mM): NaCl 125, KCl 2.5, MgSO4 1, NaH2PO4 1.25, NaHCO3 26, CaCl2 2, and D-glucose 10. Slices were maintained in ACSF and continuously bubbled with 95% O₂ and 5% CO₂, first at 34° C. for 30 min, and then at room temperature. A single slice was transferred into a submerged recording chamber and perfused constantly with carbogen-equilibrated ACSF at a rate of 2 ml/min Injected human neurons expressing RFP were visualized under a 40× water immersion objective by fluorescence and DIC optics (Carl Zeiss, Germany). Recordings were performed at 32° C. For whole-cell patch clamp studies, borosilicate glass pipettes (BF-150, Sutter Instruments, Novato, Calif., USA) with a tip resistance of 3-5 MS2 were pulled on a Flaming-Brown micropipette puller (P-1000, Sutter Instruments, Novato, Calif., USA) and filled with solution containing (in mM): K-gluconate 126, KCl 8, HEPES 20, EGTA 0.2, NaCl 2, MgATP 3, Na3GTP 0.5 (pH 7.3, 290-300 mOsmol/kg). Resting membrane potential (RMP) was recorded in current clamp mode at 0 pA immediately after establishing whole-cell configuration. Series resistance (Rseries) and input resistance (Rin) were calculated from a 5 mV pulse and monitored throughout the experiment. Unstable recordings (>10% fluctuation of Rseries value) during the course of experiments were rejected from further analysis.

For voltageclamp experiments, the membrane potential was typically held at −70 mV. Drugs were applied through a gravity-driven drug delivery system (VC-6, Warner Hamden, Conn., USA). All recordings were done using HEKA EPC10 amplifier (HEKA Elektronik, Lambrech, Germany), sampled at 10 kHz, and filtered at 2.9 kHz. Data were acquired by PatchMaster software (HEKA Elektronik, Lambrech, Germany) Na⁺ and K⁺ currents and action potentials were analyzed using Clampfit 10.5 software (Molecular devices, Palo Alto, Calif., USA). Spontaneous synaptic events were analyzed using MiniAnalysis software (Synaptosoft, Decatur, Ga., USA).

### Patch Clamp Recordings in Cell Culture

Whole-cell recordings in hiPSC-derived cell cultures were performed at 8 weeks after neuronal differentiation. Cultures were perfused at 2 ml/min at 32° C. with ACSF solution. Patch pipettes (3-5 MSΩ) were filled with a pipette solution containing (in mM): K-gluconate 126, KCl 8, HEPES 20, EGTA 0.2, NaCl2, MgATP 3, Na3GTP 0.5 (pH 7.3, 290-300 mOsmol/kg). Pipette resistance was 5-7 MΩ, and series resistance was typically 10-30 MΩ. The holding potential for voltage-clamp experiments was −70 mV. Data were collected using PatchMaster software (HEKA Elektronik, Lambrech, Germany), sampled at 10 kHz, and filtered at 2.9 kHz, then analyzed with Clampfit and Synaptosoft software.

### Equivalents

Those skilled in the art will recognize, or be able to ascertain using no more than routine experimentation, many equivalents to the specific embodiments of the invention described herein. Such equivalents are intended to be encompassed by the following claims.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of producing an adult cell from a pluripotent stem cell (PSC) comprising:
obtaining a PSC-derived cell;
contacting a PSC-derived cell with a postnatal cellular environment for a period of time;
maturing the PSC-derived cells into an adult cell, thereby producing an adult cell from a pluripotent stem cell.

2. The method of claim 1, wherein the postnatal cellular environment comprises a cell selected from the group consisting of: a cardiomyocyte, a cardiac conduction cell, a hepatocyte, a neuron, a leukocyte, an astrocyte, a brain cell, a photoreceptor cell, a retinal cell, a lung cell, a kidney cell, a pancreatic cell, a lymphocyte, a T cell, a B cell, a chondrocyte, an osteoblast, a skeletal muscle cell, a spleen cell, a stomach cell, an intestinal cell, a bladder cell and skin cell.

3. The method of claim 1, wherein the period of time is from 1 week to 8 weeks.

4. (canceled)

5. The method of claim 1, wherein the postnatal cellular environment comprises a cell from a postnatal heart, wherein the postnatal heart comprises a 2 week old postnatal heart.

6. The method of claim 5, wherein the PSC cells are contacted with the 2 week old postnatal heart via intraventricular delivery into the heart.

7. The method of claim 1, wherein the PSC comprises a human PSC.

8. The method of claim 2, wherein the adult cell is a cardiomyocyte.

9. The method of claim 1, wherein the PSC is contacted with a postnatal cellular environment from a mammal.

10. The method of claim 9, wherein the mammal is selected from a group consisting of: rodents, rats, mice, rabbits, goats, non-human primates, humans, dogs, bears, cats, lions, tigers, elephants, llamas, donkeys, mules, bovines, ovines, pigs, and horses.

11. A method of treating a disease or condition comprising administering to a subject suffering from or at risk of suffering from the disease or condition adult cells produced by the method of claim 1.

12. The method of claim 11, wherein the adult cells are generated by incubating PSCs with postnatal cellular environment tissue of the same cell tissue type as the diseased tissue for a suitable period of time until the PSCs have matured into adult cells.

13. The method of claim 11, wherein the disease is selected from a group consisting of cardiac disorder, immune disorder, cancer, gastro-intestinal disorder, neurological disorder, neurodegenerative disorder, skeletal disorder, pulmonary disorder, liver disorder, lung disorder, and pancreas disorder.

14-17. (canceled)

18. The method of claim 13, wherein the neurodegenerative disorder is selected from a group consisting of Alzheimer's disease, Parkinson's disease, amyotrophic lateral sclerosis (ALS), Huntington's disease, spinocerebellar ataxia type 1 (SCA1), and prion-disorder.

19-20. (canceled)

21. A method of treating a cardiac-related disease or condition comprising administering to a subject suffering from or at risk of suffering from a cardiac-related disease or condition an adult cell produced by the method of claim 1.

22-27. (canceled)

28. A method of treating a neurodegenerative disease or condition comprising administering to a subject suffering from or at risk of suffering from a neurodegenerative disease or condition an adult cell produced by the method of claim 1.

29-35. (canceled)

36. A composition comprising an adult cell produced by the method of claim 1.

37. A method of administering regenerative therapy to a subject comprising administering to a subject in need thereof an adult cell produced by the method of claim 1.

38. A method of diagnosing/predicting disease pathogenesis comprising detecting a biomarker of disease progression in an adult cell produced by the method of claim 1.

39. (canceled)

40. A method of diagnosing/predicting disease comprising detecting a biomarker of disease in an adult cell produced by the method of claim 1.

41. (canceled)

42. A method of determining efficacy of a candidate compound for treatment of a disease comprising
administering a candidate compound to an adult cell produced by the method of claim 1;
determining whether the disease is inhibited by the candidate compound, thereby determining treatment efficacy of the candidate compound for treatment of the disease.

43. (canceled)


SEQUENCE LISTING (46 sequences)

SEQ ID NO: 1 - DNA, 18 bp, Artificial Sequence
  Description of Artificial Sequence Synthetic primer
gacagacggcaggagcac

SEQ ID NO: 2 - DNA, 21 bp, Artificial Sequence
  Description of Artificial Sequence Synthetic primer
tctttatccttgtggctgtcc

SEQ ID NO: 3 - DNA, 21 bp, Artificial Sequence
  Description of Artificial Sequence Synthetic primer
cagaggccaactggatagatg

SEQ ID NO: 4 - DNA, 21 bp, Artificial Sequence
  Description of Artificial Sequence Synthetic primer
ggagagaaaggagatgcaggt

SEQ ID NO: 5 - DNA, 18 bp, Artificial Sequence
  Description of Artificial Sequence Synthetic primer
ggcagaagccgagttcaa

SEQ ID NO: 6 - DNA, 20 bp, Artificial Sequence
  Description of Artificial Sequence Synthetic primer
tttacctgcagagcctgtga

SEQ ID NO: 7 - DNA, 22 bp, Artificial Sequence
  Description of Artificial Sequence Synthetic primer
cagagcaatgccacctactcct

SEQ ID NO: 8 - DNA, 23 bp, Artificial Sequence
  Description of Artificial Sequence Synthetic primer
gtgaaagatgggattctcctggc

SEQ ID NO: 9 - DNA, 22 bp, Artificial Sequence
  Description of Artificial Sequence Synthetic primer
actacggtgtggcatcctgtga

SEQ ID NO: 10 - DNA, 22 bp, Artificial Sequence
  Description of Artificial Sequence Synthetic primer
ggtcctgtgaaggatgtaaggc

SEQ ID NO: 11 - DNA, 22 bp, Artificial Sequence
  Description of Artificial Sequence Synthetic primer
cctggacaaagacgagcagtgt

SEQ ID NO: 12 - DNA, 22 bp, Artificial Sequence
  Description of Artificial Sequence Synthetic primer
accactatcgctgcatcacctg

SEQ ID NO: 13 - DNA, 19 bp, Artificial Sequence
  Description of Artificial Sequence Synthetic primer
agctccccctggtagagac

SEQ ID NO: 14 - DNA, 22 bp, Artificial Sequence
  Description of Artificial Sequence Synthetic primer
accactacggagttcacgcatg

SEQ ID NO: 15 - DNA, 22 bp, Artificial Sequence
  Description of Artificial Sequence Synthetic primer
ggaccagaacacacgcttcctt

SEQ ID NO: 16 - DNA, 23 bp, Artificial Sequence
  Description of Artificial Sequence Synthetic primer
gtactgtcggtttcagaagtgcc

SEQ ID NO: 17 - DNA, 23 bp, Artificial Sequence
  Description of Artificial Sequence Synthetic primer
gaatcaagccactacagacaccg

SEQ ID NO: 18 - DNA, 23 bp, Artificial Sequence
  Description of Artificial Sequence Synthetic primer
cccagcgtctgacgtggacgagc

SEQ ID NO: 19 - DNA, 22 bp, Artificial Sequence
  Description of Artificial Sequence Synthetic primer
ttgtctcatcgacaagcgccag

SEQ ID NO: 20 - DNA, 19 bp, Artificial Sequence
  Description of Artificial Sequence Synthetic primer
ctgggaagggtctacccac

SEQ ID NO: 21 - DNA, 20 bp, Artificial Sequence
  Description of Artificial Sequence Synthetic primer
aatggcagtgtgcacgtcta

SEQ ID NO: 22 - DNA, 22 bp, Artificial Sequence
  Description of Artificial Sequence Synthetic primer
caacctggagacgcagcacaag

SEQ ID NO: 23 - DNA, 20 bp, Artificial Sequence
  Description of Artificial Sequence Synthetic primer
tgtgtccgtcgtggatctga

SEQ ID NO: 24 - DNA, 19 bp, Artificial Sequence
  Description of Artificial Sequence Synthetic primer
tttcaagcacgcctctctc

SEQ ID NO: 25 - DNA, 20 bp, Artificial Sequence
  Description of Artificial Sequence Synthetic primer
tggagggttatagcgacgat

SEQ ID NO: 26 - DNA, 27 bp, Artificial Sequence
  Description of Artificial Sequence Synthetic primer
actgtcagtgtataaagtggtgtcaat

SEQ ID NO: 27 - DNA, 18 bp, Artificial Sequence
  Description of Artificial Sequence Synthetic primer
ctttcctgccaggggttc

SEQ ID NO: 28 - DNA, 24 bp, Artificial Sequence
  Description of Artificial Sequence Synthetic primer
tgggtgggtattgtttatagtgag

SEQ ID NO: 29 - DNA, 21 bp, Artificial Sequence
  Description of Artificial Sequence Synthetic primer
tccactcgaggtaacggatag

SEQ ID NO: 30 - DNA, 22 bp, Artificial Sequence
  Description of Artificial Sequence Synthetic primer
ctgcttcttggacatccgacca

SEQ ID NO: 31 - DNA, 22 bp, Artificial Sequence
  Description of Artificial Sequence Synthetic primer
gtcacgcatcttagacaccagc

SEQ ID NO: 32 - DNA, 23 bp, Artificial Sequence
  Description of Artificial Sequence Synthetic primer
ggtgatctcacactcattggagg

SEQ ID NO: 33 - DNA, 22 bp, Artificial Sequence
  Description of Artificial Sequence Synthetic primer
taacacttgcgaagtcggcagg

SEQ ID NO: 34 - DNA, 22 bp, Artificial Sequence
  Description of Artificial Sequence Synthetic primer
ctggattgtgcggcgaaagaag

SEQ ID NO: 35 - DNA, 22 bp, Artificial Sequence
  Description of Artificial Sequence Synthetic primer
actggttgcgggtgactttgtc

SEQ ID NO: 36 - DNA, 21 bp, Artificial Sequence
  Description of Artificial Sequence Synthetic primer
ggtgaagacgcagaaaccttg

SEQ ID NO: 37 - DNA, 23 bp, Artificial Sequence
  Description of Artificial Sequence Synthetic primer
gaatcttgcagctccgatcacac

SEQ ID NO: 38 - DNA, 22 bp, Artificial Sequence
  Description of Artificial Sequence Synthetic primer
ccgacattccatgttgaggctg

SEQ ID NO: 39 - DNA, 22 bp, Artificial Sequence
  Description of Artificial Sequence Synthetic primer
atctccgccaacagcttctcct

SEQ ID NO: 40 - DNA, 22 bp, Artificial Sequence
  Description of Artificial Sequence Synthetic primer
catccctcttgagcctttcgtg

SEQ ID NO: 41 - DNA, 24 bp, Artificial Sequence
  Description of Artificial Sequence Synthetic primer
ccttcagagcgtcagagcttgctg

SEQ ID NO: 42 - DNA, 22 bp, Artificial Sequence
  Description of Artificial Sequence Synthetic primer
ctggcacattctgcctcactct

SEQ ID NO: 43 - DNA, 20 bp, Artificial Sequence
  Description of Artificial Sequence Synthetic primer
ggtgctatgttagcggcctc

SEQ ID NO: 44 - DNA, 20 bp, Artificial Sequence
  Description of Artificial Sequence Synthetic primer
ccccagccgttagtgaagag

SEQ ID NO: 45 - DNA, 22 bp, Artificial Sequence
  Description of Artificial Sequence Synthetic primer
gcttgaacaagttccgcagggt

SEQ ID NO: 46 - DNA, 21 bp, Artificial Sequence
  Description of Artificial Sequence Synthetic primer
ttgctgttgaagtcgcaggag